(19) United States
(12) Patent Application Publication
(10) Pub. No.: US 2019/0290102 A1
(43) Pub. Date: Sep. 26, 2019

(54) MEDICAL OBSERVATION APPARATUS

(71) Applicant: Sony Olympus Medical Solutions Inc., Tokyo (JP)

(72) Inventor: Toshiyuki Sasaki, Tokyo (JP)

(73) Assignee: Sony Olympus Medical Solutions Inc., Tokyo (JP)

(21) Appl. No.: 16/274,303

(22) Filed: Feb. 13, 2019

(30) Foreign Application Priority Data
Mar. 23, 2018 (JP) 2018-055482

(51) Int. Cl. A61B 1/005; A61B 1/00; A61B 34/00

(52) U.S. Cl. CPC A61B 1/0052; A61B 1/05; A61B 34/74; A61B 1/00066

(57) ABSTRACT

There is provided a medical observation apparatus, including: a medical imaging section configured to image an observation target; a support section configured to support the medical imaging section on a front end side; at least one grip detecting section installed on an outer circumference of the medical imaging section and configured to detect a grip of a user; and a control section configured to control the medical imaging section or the support section on the basis of a detection result of the grip detecting section.

### CROSS REFERENCE TO RELATED APPLICATIONS

This application claims the benefit of Japanese Priority Patent Application JP 2018-055482 filed Mar. 23, 2018, the entire contents of which are incorporated herein by reference.

### BACKGROUND

The present disclosure relates to a medical observation apparatus. In recent years, medical observation apparatus which enlarge observation targets for observation such as lesions, for example, in order to support microsurgery like neurosurgical operations are sometimes used in the medical field. As medical observation apparatus, for example, medical observation apparatus with optical microscopes and medical observation apparatus with imaging devices functioning as electronic imaging-type microscopes are exemplified. In such a medical observation apparatus, in order to support the surgery described above, high operability is necessary so that the medical observation apparatus operates as intended by a surgeon performing the surgery.

A technique in which, in order to improve operability of a microscope, a grip detecting section for detecting a grip of an operator on an operating handle for adjusting a focus is arranged, and a function of the operating handle can be switched in accordance with a grip detection result is disclosed in JP 2010-250027A.

### SUMMARY

Incidentally, in the optical medical observation apparatus and the electronic imaging medical observation apparatus, there are cases in which a medical sterilization cover called a drape is placed and used in order to secure a sterile state. Although the medical sterilization cover is a transparent or semitransparent sheet, visibility of a medical operating device of a medical observation apparatus decreases in a state in which the medical sterilization cover is placed. In this case, it is difficult for a surgeon to operate the medical observation apparatus appropriately as intended, and in some cases, it may cause a malfunction of the medical observation apparatus. Thus, in the medical observation apparatus, it is desirable that medical observation apparatus be operated with the surgeon gripping the medical operating device so that the surgeon can operate the medical observation apparatus appropriately as intended.

In the technique disclosed in the above JP 2010-250027A, in order to improve the operability, the grip detecting section is arranged in the operating handle, and the function of the operating handle is switched in accordance with the grip detection result, but it is not a technique of making a microscope operate when the operator grips the operating handle appropriately.

In this regard, the present disclosure proposes a medical observation apparatus which is novel and improved and can be operated appropriately as intended.

According to an embodiment of the present disclosure, there is provided a medical observation apparatus, including: a medical imaging section configured to image an observation target; a support section configured to support the medical imaging section on a front end side; at least one grip detecting section installed on an outer circumference of the medical imaging section and configured to detect a grip of a user; and a control section configured to control the medical imaging section or the support section on the basis of a detection result of the grip detecting section.

In addition, according to an embodiment of the present disclosure, there is provided a medical observation apparatus, including: a medical imaging section configured to image an observation target; a support section configured to support the medical imaging section on a front end side; a substantially rod-like medical operating section configured to accept an operation input for controlling the support section; at least one grip detecting section installed in the medical operating section and configured to detect a grip of a user; and a control section configured to control the medical imaging section or the support section on the basis of a detection result of the grip detecting section.

As described above, according to an embodiment of the present disclosure, it is possible to provide a medical observation apparatus which can be operated appropriately as intended.

Note that the effects described above are not necessarily limitative. With or in the place of the above effects, there may be achieved any one of the effects described in this specification or other effects that may be grasped from this specification.

### BRIEF DESCRIPTION OF THE DRAWINGS

FIG. 1 is an explanatory diagram illustrating an example of a configuration of a medical observation system according to an embodiment of the present disclosure;

FIG. 2 is an explanatory diagram illustrating an example of a use case in which the medical observation system according to the present embodiment of the present disclosure is used;

FIG. 3 is an explanatory diagram for explaining an example of the configuration of an imaging device included in a medical observation apparatus according to the present embodiment of the present disclosure (1/3);

FIG. 4 is an explanatory diagram for explaining an example of the configuration of an imaging device included in a medical observation apparatus according to the present embodiment of the present disclosure (2/3);

FIG. 5 is an explanatory diagram for explaining an example of the configuration of an imaging device included in a medical observation apparatus according to the present embodiment of the present disclosure (3/3);

FIG. 6 is a functional block diagram illustrating an example of a configuration of a medical observation apparatus according to an embodiment of the present disclosure;

FIG. 7 is a flowchart illustrating an example of a process related to a control method according to the embodiment;

FIG. 8 is an explanatory diagram illustrating an example of a display image according to the embodiment;

FIG. 9 is an explanatory diagram for describing an example of a configuration of an imaging device included in a medical observation apparatus according to a first modified example of the embodiment;

FIG. 10 is an explanatory diagram for describing another example of the configuration of the imaging device included in the medical observation apparatus according to the first modified example of the embodiment;

FIG. 11 is an explanatory diagram illustrating an example of a display image according to the first modified example of the embodiment;

FIG. 12 is an explanatory diagram for describing an example of a configuration of an imaging device included in a medical observation apparatus according to a second modified example of the embodiment;

FIG. 13 is an explanatory diagram for describing a third modified example of the embodiment;

FIG. 14 is a flowchart illustrating an example of a process related to a control method according to the third modified example of the embodiment;

FIG. 15 is a flowchart illustrating an example of a process related to a control method according to the fourth modified example of the embodiment; and

FIG. 16 is an explanatory diagram for describing an example of a configuration of a medical operating device of a medical observation apparatus according to a fifth modified example of the embodiment.

### DETAILED DESCRIPTION OF THE EMBODIMENT(S)

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Further, in this specification and the drawings, a plurality of constituent elements having substantially the same or similar functional configuration may be distinguished by adding different letters after the same reference numerals. However, in a case in which it is not necessary to particularly distinguish each of a plurality of constituent elements having substantially the same or similar functional configuration, only the same reference numerals are attached. Further, similar components of different embodiments may be distinguished by adding different letters after the same reference numerals. However, in a case in which it is not necessary to particularly distinguish each of similar components, only the same reference numerals are attached.

Further, the drawings to be referred to in the following description are for describing an embodiment of the present disclosure and for facilitating understanding thereof, and shapes, sizes, ratios, and the like illustrated in the drawings may be different from actual ones in order to facilitate understanding thereof. Further, description of specific shapes in the following description does not refer only to cases in which the corresponding shapes coincide with geometrical shapes, but also refers to a tolerable difference in manufacture and use of a medical observation apparatus, and shapes similar to corresponding shapes are included. For example, in the following description, a case in which a “circular shape” or “substantially circular shape” is described is not limited to a perfect circle and may refer to a shape similar to a perfect circle such as an ellipse shape.

Further, the description will proceed in the following order.

1. Schematic configuration of media observation system
2. Schematic configurations of apparatuses of media observation system
2.1. Medical observation apparatus 100

### 2.1.1. Base 102

### 2.1.2. Arm 104

2.1.3. Imaging device 106
2.2. Display apparatus 200
3. Background to creation of embodiment of the present disclosure

### 4. Embodiment

4.1. Detailed configuration of medical observation apparatus 100
4.1.1. Grip detecting section 140
4.1.2. Light emitting section 150
4.1.3. Control section 170
4.2. Control method according to the present embodiment
5. Modified examples
5.1. First modified example
5.2. Second modified example
5.3. Third modified example
5.4. Fourth modified example
5.5. Fifth modified example

### 6. Conclusion

### 7. Supplement

### 1. SCHEMATIC CONFIGURATION OF MEDICAL OBSERVATION SYSTEM

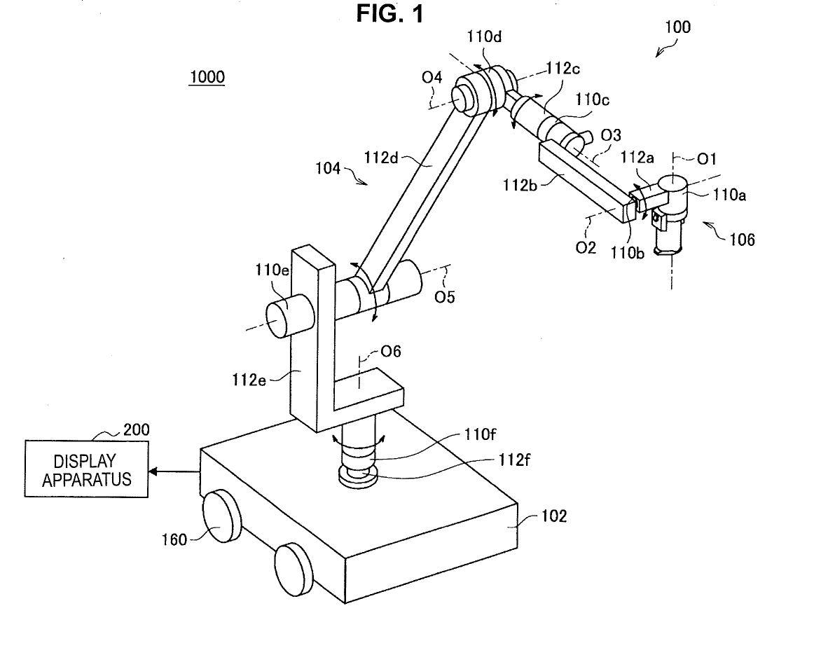

First, before describing an embodiment of the present disclosure in detail, an overview of an example of a configuration of a medical observation system according to an embodiment of the present disclosure will be described with reference to FIG. 1. FIG. 1 is an explanatory diagram illustrating an example of a configuration of a medical observation system 1000 according to the present embodiment. Further, in the following description, a medical observation apparatus 100 according to the present embodiment will be described using an example in which the medical observation apparatus 100 according to the present embodiment is an electronic imaging medical observation apparatus as a main example, but the medical observation apparatus 100 according to the present embodiment is not limited to an electronic imaging medical observation apparatus. For example, the medical observation apparatus 100 according to the present embodiment may be an optical medical observation apparatus.

As illustrated in FIG. 1, the medical observation system 1000 includes, for example, a medical observation apparatus 100 and a display apparatus (display section) 200. Further, the medical observation system 1000 according to the present embodiment is not limited to the example illustrated in FIG. 1. For example, although not illustrated in FIG. 1, the medical observation system 1000 according to the present embodiment may further include a control device that controls various kinds of operations in the medical observation apparatus 100. As the control device, for example, an arbitrary device capable of performing a process related to a control method according to the present embodiment to be described later such as a “processor,” a “medical controller,” or a “computer such as a server” may be used. Further, the control device may be realized by, for example, an integrated circuit (IC) which can be incorporated into a function of the device as described above.

Further, the medical observation system 1000 according to the present embodiment may have a configuration including a plurality of medical observation apparatuses 100 and a plurality of display apparatuses 200. Further, in a case in which the medical observation system 1000 according to the present embodiment includes a plurality of medical observation apparatuses 100 and a plurality of display apparatuses 200, the medical observation apparatuses 100 and the display apparatuses 200 may be associated in a one to one manner. Alternatively, in the above case, a plurality of medical observation apparatuses 100 may be associated with one display apparatus 200. Further, in a case in which a plurality of medical observation apparatuses 100 are associated with one display apparatus 200, for example, when a switching operation or the like is performed, display of a medical captured image captured by the medical observation apparatus 100 (the details thereof will be described later) can be switched in the display apparatus 200.

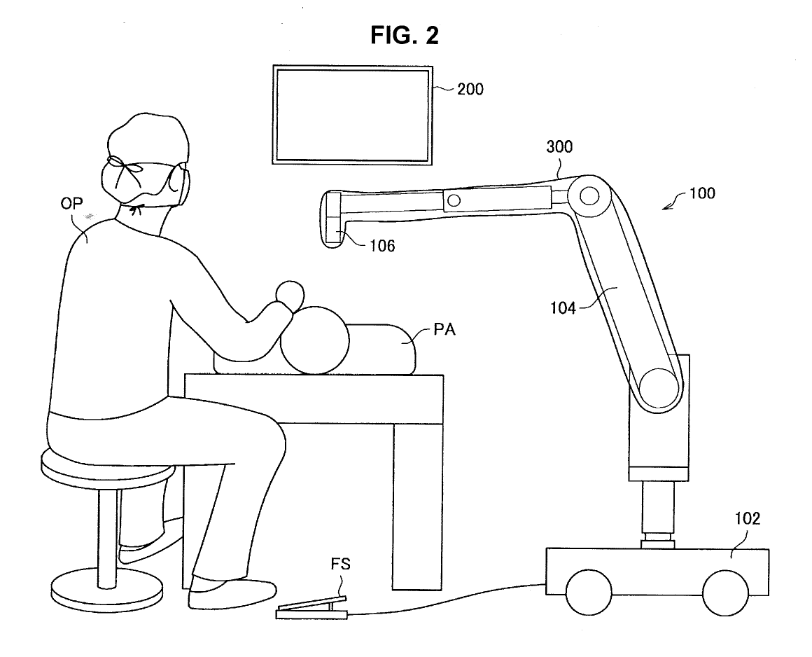

FIG. 2 is an explanatory diagram illustrating an example of a use case in which the medical observation system 1000 according to the present embodiment is used. An example of a use case in which the medical observation system 1000 according to the present embodiment is used will be described below with reference to FIG. 2. As illustrated in FIG. 2, a patient PA (a patient who is a subject receiving medical treatment) serving as an observation target is imaged by an imaging device 106 (medical imaging section) (details will be described later) included in the medical observation apparatus 100. In the following description, a captured image captured by the medical observation apparatus 100 according to the present embodiment such as a captured image obtained by imaging the patient PA who is a subject receiving medical treatment is referred to as a “medical captured image.” Further, the medical captured image captured by the medical observation apparatus 100 is displayed on a display screen of the display apparatus 200. Further, a surgeon OP performing medical treatment using the medical observation apparatus 100 (an example of a user of the medical observation apparatus 100) can perform medical treatment such as a surgery on the patient PA while viewing the medical captured image displayed on the display screen of the display apparatus 200.

Further, the surgeon OP can cause an arm 104 (support section) (details will be described later) or the like of the medical observation apparatus 100 to operate so that the medical observation apparatus 100 can enter a desired state by operating a medical operating device outside the medical observation apparatus 100 such as a foot switch FS or a medical operating device (medical operating section) (not illustrated) of the medical observation apparatus 100.

Further, as illustrated in FIG. 2, in the medical observation apparatus 100, there are cases in which a medical sterilization cover (drape) 300 is placed in order to secure a sterile state. In detail, the medical sterilization cover 300 is formed of a transparent or translucent material and covers at least a part of the imaging device 106 or the arm 104 of the medical observation apparatus 100 to thereby secure the sterile state of the medical observation apparatus 100.

### 2. SCHEMATIC CONFIGURATIONS OF APPARATUSES OF MEDICAL OBSERVATION SYSTEM

The overview of the example of the configuration of the medical observation system 1000 according to an embodiment of the present disclosure has been described above. Next, schematic configurations of the apparatuses constituting the medical observation system 1000 will be sequentially described.

### 2.1. Medical Observation Apparatus 100

First, the medical observation apparatus 100 according to the present embodiment will be described. Here, the medical observation apparatus 100 is described as an electronic imaging medical observation apparatus. For example, in a case in which the medical observation apparatus 100 is used at the time of surgery, the surgeon (an example of the user of the medical observation apparatus 100) OP can perform various types of treatments on an operating site in accordance with a surgical procedure while observing the operating site with reference to the medical captured image which is captured by the medical observation apparatus 100 and displayed on the display screen of the display apparatus 200.

An example of a hardware configuration of the medical observation apparatus 100 according to the present embodiment will be described with reference to FIG. 1. As illustrated in FIG. 1, the medical observation apparatus 100 includes, for example, a base 102, an arm 104, and an imaging device 106 (medical imaging section). The respective parts of the hardware configuration of the medical observation apparatus 100 will be sequentially described below in detail. Further, the hardware configuration of the medical observation apparatus 100 according to the present embodiment is not limited to the configuration illustrated with reference to FIG. 1.

### (2.1.1. Base 102)

The base 102 is a base of the medical observation apparatus 100, and one end of an arm 104 to be described later is connected to the base 102, and supports the arm 104 and the imaging device 106. Further, for example, casters 160 are installed in the base 102, and the medical observation apparatus 100 is grounded on a floor surface via the casters 160. Since the casters 160 are installed, the medical observation apparatus 100 can easily move on the floor surface by the casters 160. Further, the medical observation apparatus 100 according to the present embodiment may have a configuration in which the arm 104 is directly attached to a ceiling, a wall, or the like of an operating room or the like without including the base 102. For example, in a case in which the arm 104 is directly attached to the ceiling, the medical observation apparatus 100 is configured such that the arm 104 is hung from the ceiling.

### (2.1.2. Arm 104)

The arm 104 is constituted by connecting a plurality of links 112 to one another via joint sections 110. Further, the arm 104 supports the imaging device 106 to be described later on a front end side. The imaging device 106 supported by the arm 104 can move space three-dimensionally, and a position and an attitude of the imaging device 106 after the movement is fixed by the arm 104.

More specifically, as illustrated in FIG. 1, the arm 104 includes, for example, multiple joint sections 110a, 110b, 110c, 110d, 110e, and 110f, and multiple links 112a, 112b, 112c, 112d, 112e, and 112f rotatably joined to each other by the joint sections 110a, 110b, 110c, 110d, 110e, and 110f. The rotatable range of each of the joint sections 110a, 110b, 110c, 110d, 110e, and 110f may be set arbitrarily during the design stage, the manufacturing stage, or the like so that the desired motion of the arm 104 is realized.

In other words, in the medical observation apparatus 100 illustrated in FIG. 1, six degrees of freedom are realized in relation to the movement of the imaging device 106 by six rotation axes (first axis O1, second axis O2, third axis O3, fourth axis O4, fifth axis O5, and sixth axis O6) corresponding to the six joint sections 110a, 110b, 110c, 110d, 110e, and 110f included in the arm 104. More specifically, in the medical observation apparatus 100 illustrated in FIG. 1, motion with six degrees of freedom, including three degrees of translational freedom and three degrees of rotational freedom, is realized.

Each of the joint sections 110a, 110b, 110c, 110d, 110e, and 110f of the arm 104 are provided with a brake (not illustrated) that restrains rotation in each of the joint sections 110a, 110b, 110c, 110d, 110e, and 110f. The brake according to the present embodiment may be a brake of an arbitrary method, such as a mechanically driven brake or an electrically driven electromagnetic brake, for example. The driving of the above brakes can be controlled by, for example, a processor that functions as the control section 170 described later, or an external medical control apparatus (not illustrated). By controlling the driving of the above brakes, in the medical observation apparatus 100, the operating mode of the arm 104 can be set. Examples of operating modes of the arm 104 can include a locked mode and a free mode.

Herein, the locked mode according to the present embodiment is, for example, an operating mode in which the position and the attitude of the imaging device 106 are locked by using the above-described brakes to restrain rotation about each rotation axis provided in the arm 104. By having the arm 104 enter the locked mode, the operating state of the medical observation apparatus 100 becomes a locked state in which the position and the attitude of the imaging device 106 are locked. Also, the free mode according to the present embodiment is, for example, an operating mode in which the above brakes are released, thereby allowing each rotation axis provided in the arm 104 to rotate freely. For example, in the free mode described above, the position and the attitude of the imaging device 106 are adjustable by direct operations performed by the surgeon OP. Herein, a direct operation according to the present embodiment means, for example, an operation in which the surgeon OP grips the imaging device 106 with his or her fingers, and directly moves the imaging device 106.

Further, in the present embodiment of the present disclosure, for example, actuators (not illustrated) are provided in each of the joint sections 110a, 110b, 110c, 110d, 110e, and 110f. In this case, each of the joint sections 110a, 110b, 110c, 110d, 110e, and 110f rotates about the corresponding rotation axis by the driving of the actuators. The driving of the actuators is controlled by, for example, a processor that functions as the control section 170 described later, or an external medical control apparatus (not illustrated). By having each of the joint sections 110a, 110b, 110c, 110d, 110e, 110f rotate about the corresponding rotation axis by the driving of the actuators, various operations of the arm 104, such as extending and contracting (folding up) the arm 104, for example, are realized.

Specifically, the joint section 110a has an approximately cylindrical shape, and supports the imaging device 106 (the top end of the imaging device 106 in FIG. 1) on the front end portion of the joint section 110a (the bottom end portion in FIG. 1), so as to allow revolution about a rotation axis (first axis O1) parallel to the central axis of the imaging device 106. Herein, the medical observation apparatus 100 is configured so that the first axis O1 is aligned with the optical axis in the imaging device 106. In other words, by causing the imaging device 106 to revolve about the first axis O1 illustrated in FIG. 1, the medical captured image captured by the imaging device 106 can be changed so that the field of view rotates.

The link 112a is an approximately rod-shaped member, and securely supports the joint section 110a. The link 112a extends in a direction orthogonal to the first axis O1, for example, and is connected to the joint section 110b.

The joint section 110b has an approximately cylindrical shape, and supports the link 112a so as to allow revolution about a rotation axis (second axis O2) orthogonal to the first axis O1. Also, the link 112b is securely connected to the joint section 110b.

The link 112b is an approximately rod-shaped member, and extends in a direction orthogonal to the second axis O2. Also, each of the joint section 110b and the joint section 110c is connected to the link 112b.

The joint section 110c has an approximately cylindrical shape, and supports the link 112b so as to allow revolution about a rotation axis (third axis O3) mutually orthogonal to each of the first axis O1 and the second axis O2. Also, one end of the link 112c is securely connected to the joint section 110c.

Herein, by having the front end side (the side on which the imaging device 106 is provided) of the arm 104 revolve about the second axis O2 and the third axis O3, the imaging device 106 can be made to move so that the position of the imaging device 106 in the horizontal plane is changed. In other words, in the medical observation apparatus 100, controlling the rotation about the second axis O2 and the third axis O3 makes it possible to move the field of view of the medical captured image in a flat plane.

The link 112c is a member in which one end has an approximately cylindrical shape, and the other end has an approximately rod-like shape. On the side of the one end of the link 112c, the joint section 110c is securely connected so that the central axis of the joint section 110c and the central axis of the approximately cylindrical shape are the same. Also, on the side of the other end of the link 112c, the joint section 110d is connected.

The joint section 110d has an approximately cylindrical shape, and supports the link 112c so as to allow revolution about a rotation axis (fourth axis O4) orthogonal to the third axis O3. The link 112d is securely connected to the joint section 110d.

The link 112d is an approximately rod-shaped member, and extends orthogonally to the fourth axis O4. One end of the link 112d is securely connected to the joint section 110d so as to abut the approximately cylindrical side face of the joint section 110d. Also, the joint section 110e is connected to the other end of the link 112d (the end on the opposite side of the side where the joint section 110d is connected).

The joint section 110e has an approximately cylindrical shape, and supports one end of the link 112d so as to allow revolution about a rotation axis (fifth axis O5) parallel to the fourth axis O4. Also, one end of the link 112e is securely connected to the joint section 110e.

Herein, the fourth axis O4 and the fifth axis O5 are rotation axis about which the imaging device 106 may be moved in the vertical direction. By having the front end side (the side on which the imaging device 106 is provided) of the arm 104 revolve about the fourth axis O4 and the fifth axis O5, the position of the imaging device 106 in the vertical direction changes. Thus, by having the front end side (the side on which the imaging device 106 is provided) of the arm 104 revolve about the fourth axis O4 and the fifth axis O5, changing the distance between the imaging device 106 and an observation target, such as an operating site of a patient, becomes possible.

The link 112e is a member that includes a combination of a first member having an approximate L-shape with one edge extending in the vertical direction while the other edge extends in the horizontal direction, and a rod-like second member that extends vertically downward from the part of the first member that extends in the horizontal direction. The joint section 110e is securely connected to the part of the first member of the link 112e that extends in the vertical direction. Also, the joint section 110f is connected to the second member of the link 112e.

The joint section 110f has an approximately cylindrical shape, and supports the link 112e so as to allow revolution about a rotation axis (sixth axis O6) parallel to the vertical direction. Also, the link 112f is securely connected to the joint section 110f.

The link 112f is an approximately rod-shaped member, and extends in the vertical direction. The joint section 110f is connected to one end of the link 112f. Also, the other end of the link 112f (the end on the opposite side of the side where the joint section 110f is connected) is securely connected to the base 102.

As described above, since the arm 104 has the above-described configuration, six degrees of freedom regarding the movement of the imaging device 106 are realized in the medical observation apparatus 100. Further, in the present embodiment, the configuration of the arm 104 is not limited to the above-described configuration. For example, although FIG. 1 illustrates an example configured so that six degrees of freedom are realized with respect to the driving of the imaging device 106, the configuration of the arm 104 is not limited to a configuration whereby the degrees of freedom with respect to the driving of the imaging device 106 become six degrees of freedom. For example, it is sufficient to configure the arm 104 so that the imaging device 106 can move appropriately in accordance with the application, and factors such as the number and arrangement of joint sections 110 and links 112, and the directions of the drive shafts of the joint sections 110 can be set appropriately so that the arm 104 has the desired degrees of freedom.

### (2.1.3. Imaging Device 106)

The imaging device 106 can be supported by the arm 104, and images an observation target such as an operating site of a patient PA, for example. Imaging in the imaging device 106 can be controlled by, for example, a processor that functions as the control section 170 described later, or an external medical control apparatus (not illustrated). Here, the imaging device 106 is described as having, for example, a configuration corresponding to an electronic imaging microscope.

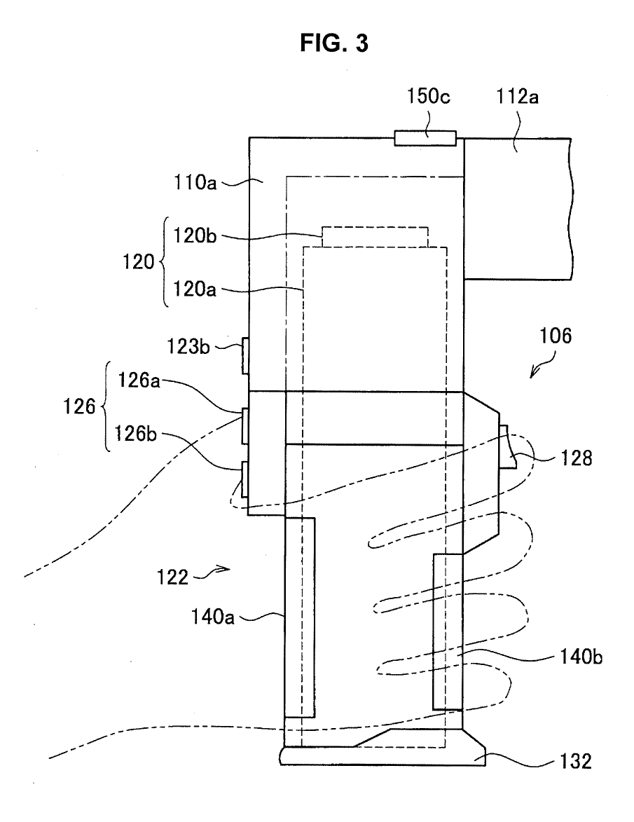

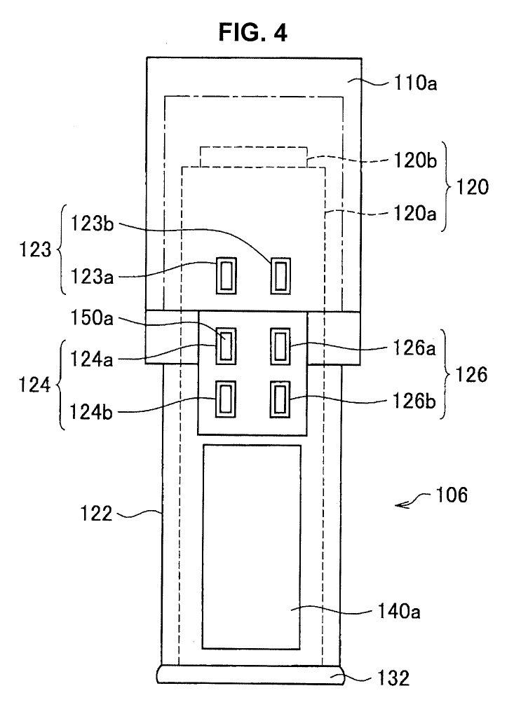

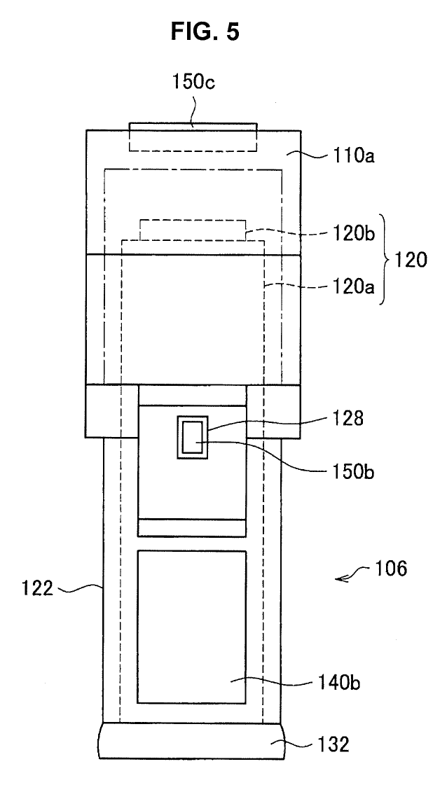

The imaging device 106 according to the present embodiment will be described with reference to FIGS. 3 to 5. FIGS. 3 to 5 are explanatory diagrams for describing an example of a configuration of the imaging device 106 included in the medical observation apparatus 100 according to the present embodiment. FIG. 3 is a side view of the imaging device 106, FIG. 4 is a front view of the imaging device 106, and FIG. 5 is a rear view of the imaging device 106. Further, in the following description, it is assumed that a side of the imaging device 106 on which a function switch 123, a zoom switch 124, and a focus switch 126 are installed is a front side, and a side of the imaging device 106 on which an operating mode change switch 128 is installed is a back side. However, the definitions of the front and the back of the imaging device 106 are for convenience for describing the present embodiment, and the definitions of the front and the back of the imaging device 106 are not limited thereto. Further, in the following description, unless set forth otherwise, upward and downward directions of the imaging device 106 are defined such that an opening surface side at a lower end of the imaging device 106 (specifically, a barrel member 122) is a lower side, and a side opposite to the opening surface side is an upper side in FIG. 1 and FIGS. 3 to 5. Accordingly, the upward and downward directions of the imaging device 106 may be different from the upward and downward directions according to actual gravitational acceleration.

In more detail, the imaging device 106 includes, as illustrated in FIGS. 3 to 5, for example, an imaging member 120 and a barrel member 122 having an approximately cylindrical shape, with the imaging member 120 being provided inside the barrel member 122.

On an aperture on the bottom end of the barrel member 122 (the lower end in FIGS. 3 to 5), for example, a cover glass (not illustrated) for protecting the imaging member 120 is provided. Additionally, for example, a light source (not illustrated) is provided inside the barrel member 122, and during imaging, the subject is irradiated with illuminating light radiating from the light source through the cover glass described above. Reflected light (observation light) from the subject irradiated with illuminating light enters the imaging member 120 through the cover glass, whereby an image signal indicating the subject (an image signal indicating a captured image) can be obtained by the imaging member 120.

As the imaging member 120, any of various known types of configurations used in an electronic imaging microscope section can be applied. The imaging member 120 includes an optical system 120a and an image sensor 120b including an imaging element that takes an image of an observation target with light transmitted through the optical system 120a, for example. The optical system 120a includes optical elements such as a mirror and one or multiple lenses, such as an objective lens, a zoom lens, and a focus lens, for example. Examples of the image sensor 120b can include an image sensor using multiple imaging elements, such as a complementary metal-oxide semiconductor (CMOS) or a charge-coupled device (CCD).

The imaging member 120 may also include a pair of imaging elements, or in other words, be configured to function as what is called a stereo camera. The imaging member 120 is equipped with one or multiple functions typically provided in an electronic imaging microscope section, including at least a zoom function (one or both of an optical zoom function and an electronic zoom function), such as an autofocus (AF) function.

In addition, the imaging member 120 may also be configured to be capable of imaging at what are called high resolutions, such as 4K and 8K, for example. By configuring the imaging member 120 to be capable of imaging at high resolutions, it becomes possible to ensure a predetermined resolution (such as full HD image quality, for example), while also displaying a medical captured image or the like on the display apparatus 200 having a large display screen, such as 50 inches or more, for example. As a result, visibility of the medical captured image or the like is improved for the surgeon OP watching the display screen. Also, by configuring the imaging member 120 to be capable of imaging at high resolutions, even if the medical captured image is enlarged by the electronic zoom function and displayed on the display screen of the display apparatus 200, it is still possible to ensure a predetermined resolution. Furthermore, in the case of using the electronic zoom function to ensure a predetermined resolution, since it is possible to reduce the performance of the optical zoom function in the imaging device 106, the optical system of the imaging device 106 can be simplified, and the imaging device 106 can be more compact.

Further, in the following description, an observation direction of the imaging device 106 is a direction along a straight line extending from an imaging member 120 installed in the barrel member 122 of the imaging device 106 to a subject (observation target) via a cover glass (not illustrated).

Further, for example, various types of medical operating devices for controlling an operation of the imaging device 106 are installed in the imaging device 106. For example, as illustrated in FIGS. 3 to 5, in the imaging device 106, the function switch (operating button) 123, the zoom switch (operating button) 124, the focus switch (operating button) 126, and the operating mode change switch (operating button) 128 are installed on an outer circumference of the barrel member 122 of the imaging device 106. Further, in the present embodiment, a position at which the zoom switch 124, the focus switch 126, and the operating mode change switch 128 are installed and shapes thereof are not limited to the example illustrated in FIGS. 3 to 5. Further, the medical operating device according to the present embodiment is not limited to the function switch 123, the zoom switch 124, the focus switch 126, and the operating mode change switch 128.

The function switch 123 (specifically, function switches 123a and 123b) is an example of the medical operating device for executing the functions of the imaging device 106 and a control section 170 to be described later and is installed on an outer circumference of the front side and the upper end side of the barrel member 122 of the imaging device 106, for example, as illustrated in FIGS. 3 and 4. More specifically, the function switch 123 is used to execute a function arbitrarily assigned by the surgeon OP and is assigned, for example, an autofocusing operation of the imaging device 106, a function of changing a frequency of illumination light irradiated from a light source, and the like. The zoom switch 124 and the focus switch 126 are an example of a medical operating device for adjusting imaging conditions in the imaging device 106 and are installed on an outer circumference of the front side and the upper end side of the barrel member 122 of the imaging device 106, for example, as illustrated in FIGS. 3 and 4. More specifically, the zoom switch 124 includes, for example, a zoom-in switch 124a for increasing a zoom magnification (enlargement magnification) and a zoom-out switch 124b for reducing the zoom magnification. When the operation on the zoom switch 124 is performed, the zoom magnification is adjusted, and the zoom in the imaging device 106 is changed. The focus switch 126 includes, for example, a long-distance view focus switch 126a for increasing a focal distance to the observation target (subject) and a short-distance view focus switch 126b for decreasing the focal distance to the observation target. When the operation on the focus switch 126 is performed, the focal length is adjusted, and the focus in the imaging device 106 is changed.

The operating mode change switch 128 is an example of a medical operating device for changing an operating mode of the arm 104 in the imaging device 106 and is installed on an the outer circumference on the back side and the upper end side of the barrel member 122 of the imaging device 106, for example, as illustrated in FIGS. 3 and 5. When an operation input is performed on the operating mode change switch 128, the operating mode of the arm 104 is changed. Examples of the operating mode of the arm 104 include the locked mode and the free mode as described above. In more specifically, one example of an operation input with respect to the operating mode change switch 128 is an operation of pressing the operating mode change switch 128. For example, the operating mode of the arm 104 may be set to the free mode while the surgeon OP is pressing the operating mode change switch 128, and the operating mode of the arm 104 may be set to the locked mode when the surgeon OP is not pressing the operating mode change switch 128.

For example, as illustrated by a line in FIG. 3 that alternates between long dashes and pairs of short dashes, the surgeon OP can adjust the position and the attitude of the imaging device 106 by gripping the lower side of the barrel member 122 (a lower end portion of the barrel member 122 in FIG. 3) while pushing the operating mode change switch 128 and performing an operation of moving the imaging device 106 directly.

Further, in the present embodiment, in order to move the imaging device 106 more safely as intended by the surgeon OP, the operating mode of arm 104 is set to the free mode only in a case in which it is detected that the surgeon OP grips the barrel member 122 appropriately in addition to the operation input to the operating mode change switch 128. In this regard, in the present embodiment, the grip detecting section 140 for detecting the grip of the surgeon OP is installed on the outer circumference of the barrel member 122. Further, in the present embodiment, a light emitting section 150 capable of turning on a light is installed in the zoom switch 124 or the like so that the position or the like of the function switch 123, the zoom switch 124, the focus switch 126, and the operating mode change switch 128 can be easily recognized by the surgeon OP. Further, the grip detecting section 140 and the light emitting section 150 according to the present embodiment will be described later in detail.

Note that, although FIGS. 3 and 5 illustrate an example in which various types of medical operating devices for controlling the operation of the imaging device 106 are provided on the imaging device 106, some or all of the medical operating devices illustrated in FIGS. 3 and 5 may also not be provided on the imaging device 106. To give one example, the various types of medical operating devices for controlling the operation of the imaging device 106 may also be provided in another part other than the imaging device 106 included in the medical observation apparatus 100 according to the present embodiment. Also, to give another example, the various types of medical operating devices for controlling the operation of the imaging device 106 may also be external medical operating devices, such as a footswitch FS or a remote controller.

Further, in the imaging device 106, for example, a protruding member 132 may be formed in the vicinity of a lower end of the barrel member 122 in order to further improve operability, convenience, or the like when the surgeon OP performing an operation on various types of medical operating devices performs an operation. The protruding member 132 can prevent a hand from blocking a field of view of an optical system 120a or prevent the cover glass (not illustrated) from being contaminated by a hand touching the cover glass when the surgeon OP operates the barrel member 122 with a hand. Further, in the present embodiment, the position at which the protruding member 132 is formed and the shape of the protruding member 132 are not limited to the example illustrated in FIGS. 3 to 5.

Further, an image signal (image data) generated by imaging in the imaging device 106 undergoes, for example, image processing in a processor functioning as a control section 170 to be described later. As the image processing, for example, one or more processes among various types of processes such as gamma correction, white balance adjustment, enlargement or reduction of an image related to an electronic zoom function, and inter-pixel correction can be used. Further, the medical observation apparatus 100 transmits, for example, a display control signal and the image signal which has undergone the image processing to the display apparatus 200 to be described later. As the display control signal and the image signal are transmitted to the display apparatus 200, the medical screen captured image in which the observation target is imaged (for example, the captured image in which the operating site is imaged) can be enlarged or reduced with a desired magnification by either or both of an optical zoom function and an electronic zoom function and displayed on the display screen of the display apparatus 200. Further, in a case in which the medical observation system 1000 according to the present embodiment includes a medical control apparatus (not illustrated) for controlling various types of operations in the medical observation apparatus 100, the image processing may be performed in the medical control apparatus.

Further, although not illustrated in FIG. 1, the medical observation apparatus 100 may include one or more processors (not illustrated) which are constituted by an arithmetic circuit such as a micro processing unit (MPU) (not illustrated), a read only memory (ROM) (not illustrated), a random access memory (RAM) (not illustrated), a recording medium (not illustrated), a communication device (not illustrated), and an input device (not illustrated). Further, the medical observation apparatus 100 can be driven by, for example, electric power supplied from an internal power source such as a battery included in the medical observation apparatus 100, electric power supplied from an external power source connected thereto, or the like.

The processor (not illustrated) functions as the control section 170 to be described later. Further, the control section 170 according to the present embodiment will be described later in detail.

The ROM (not illustrated) stores a program used by a processor (not illustrated) or control data such as an operation parameter. Further, the RAM (not illustrated) temporarily stores a program executed by the processor or the like. Further, the recording medium (not illustrated) stores various data such as control data and various types of applications. Here, examples of the recording medium include a magnetic recording medium such as a hard disk and a non-volatile memory such as a flash memory. Further, the recording medium may be detachable from the medical observation apparatus 100.

The communication device (not illustrated) is a communication device included in the medical observation apparatus 100 and plays a role of performing wireless or wired communication with an external apparatus such as the display apparatus 200. Here, examples of the communication device include an IEEE 802.15.1 port and a transmission/reception circuit (wireless communication), an IEEE 802.11 port and a transmission/reception circuit (wireless communication), a communication antenna and a radio frequency (RF) circuit (wireless communication), and a local area network (LAN) terminal and a transmission/reception circuit (wired communication). For example, communication in the communication device can be controlled by the control section 170 to be described later.

The input device (not illustrated) is an interface for inputting information to the medical observation apparatus 100, and can be realized by, for example, a keyboard, a mouse, a touch panel overlapped with the display apparatus 200 to be described later, or the like.

### <2.2 Display Apparatus 200>

The display apparatus 200 is a display device in the medical observation system 1000, and corresponds to an external display device from the perspective of the medical observation apparatus 100. The display apparatus 200 displays various images on a display screen, such as a medical captured image (a moving image or multiple still images; the same applies hereinafter) taken in the medical observation apparatus 100, or an image related to a user interface (UI), for example. In addition, the display apparatus 200 may also be a configuration capable of 3D display. The display on the display apparatus 200 is controlled by, for example, the control section 170 or the medical observation apparatus (not illustrated).

In the medical observation system 1000, the display apparatus 200 is installed in an arbitrary location visible to a person involved in a surgery OP or the like inside an operating room, such as on a wall, the ceiling, or the floor of the operating room, for example. Examples of the display apparatus 200 include a liquid crystal display, an organic electro-luminescence (EL) display, a cathode ray tube (CRT) display, and the like. Note that the display apparatus 200 is not limited to the example illustrated above. For example, the display apparatus 200 may also be an arbitrary wearable apparatus that is used by being worn on the body of the surgeon OP or the like, such as a head-mounted display, an eyewear-type apparatus, or the like.

In addition, the display apparatus 200 can run on electric power supplied from an internal power source such as a battery provided in the display apparatus 200, on electric power supplied from a connected external power source, or the like, for example.

### 3. BACKGROUND TO CREATION OF EMBODIMENT OF THE PRESENT DISCLOSURE

Before describing an embodiment of the present disclosure in further detail, the background in which the present inventor ended up with creation of an embodiment of the present disclosure will be described.

As described above, the medical observation apparatus 100 may be used in a state in which the medical sterilization cover 300 is placed in order to secure the sterile state. In the state in which the medical sterilization cover 300 is placed, the medical sterilization cover 300 is a transparent or semitransparent sheet, but the visibility of the medical operating devices of the medical observation apparatus 100 (for example, the function switch 123, the zoom switch 124, the focus switch 126, and the operating mode change switch 128) is lowered since they are shielded by the medical sterilization cover 300. In this case, it is difficult for the surgeon OP to operate the medical observation apparatus 100 appropriately as intended, and in some cases, the medical observation apparatus 100 may malfunction.

Further, there are cases in which the observation target such as the operating site of the patient PA is illuminated, the whole operating room is darkened, and the medical observation apparatus 100 is used. In this case, since the surgeon OP is unable to directly view the medical operating device, it is difficult to appropriately operate the medical operating device.

In addition, there are cases in which the surgeon OP attempts to operate the medical operating device without turning his/her eyes to the medical operating device while keeping his eyes on the operating site or the display apparatus 200 displaying the medical captured image of the operating site. Further, positions of various types of medical operating devices or functions assigned to various types of medical operating devices often differ depending on function assignment by the surgeon OP, a type of medical observation apparatus, a model number, a manufacturing company, or the like. Therefore, in a case in which the surgeon OP attempts to operate the medical operating device without turning his/her eyes to the medical operating device, since the surgeon OP does not remember the positions or the functions of the medical operating devices of each medical observation apparatus in detail, the surgeon OP may not be able to operate the medical operating devices appropriately.

More specifically, in the medical observation apparatus 100, the surgeon OP can perform an operation of moving the imaging device 106 directly by gripping the lower side of the barrel member 122 while pushing the operating mode change switch 128, for example, as illustrated by a line in FIG. 3 that alternates between long dashes and pairs of short dashes. Further, in the medical observation apparatus 100, the surgeon OP can adjust the imaging condition in the imaging device 106 by operating the function switch 123, the zoom switch 124, or the focus switch 126.

However, as described above, in a case in which the visibility of the operating mode change switch 128 is lowered by the medical sterilization cover 300, the surgeon OP may not be able to perform an appropriate operation of gripping the lower side of the barrel member 122 while pushing the operating mode change switch 128. In this case, since the position and attitude of the imaging device 106 are unable to be adjusted, the progress of the surgery by the surgeon OP may be hindered.

Further, when the imaging conditions in the imaging device 106 is adjusted, in a case in which the visibility of the zoom switch 124 or the like is lowered by the medical sterilization cover 300, the surgeon OP may erroneously push the operating mode change switch 128 other than the zoom switch 124 or the like. In this case, a malfunction in which the operating mode of the arm 104 is set to the free mode, the imaging device 106 is moved due to the pressure from the hand of the surgeon OP, and the observation range of the imaging device 106 is moved contrary to the intention of the surgeon OP is assumed to occur. Further, in a case in which the pressure is applied to the operating mode change switch 128 or the like due to the tension of the medical sterilization cover 300, a malfunction is assumed to occur as well, similarly to the above case.

Further, in a case in which the surgeon OP is unable to directly recognize the operating mode change switch 128 or the like as the whole operating room is darkened or in a case in which the surgeon OP attempts operate the zoom switch 124 or the like while keeping his/her eyes on the operating site or the display apparatus 200 displaying the medical captured image of the operating site, a malfunction is assumed to occur as well, similarly to the above case.

Further, the surgery may be performed using a plurality of medical apparatuses by a plurality of surgeons OP or a plurality of assistants supporting the surgeon OP. In this case, the pressure is applied to the operating mode change switch 128 or the like by a part of body of a plurality of surgeons OP or a plurality of assistants or some of the medical apparatuses, a malfunction is assumed to occur as well, similarly to the above case.

As described above, there are situations in which it is difficult for the surgeon OP to operate the medical observation apparatus 100 appropriately as intended. In view of such situations, the present inventor has conducted keen examination in order to obtain a medical observation apparatus which can prevent the malfunctions described above, that is, can be operated appropriately as intended. In such examination, the present inventor obtained an ideal of receiving the operation input to the medical operating device in a case in which it is detected that the surgeon OP appropriately grips the medical operating device or the like in order to operate the medical observation apparatus 100 appropriately as intended while preventing the malfunctions described above. Further, the present inventor ended up with an embodiment of the present disclosure to be described below on the basis of such an idea.

In other words, in an embodiment of the present disclosure created by the present inventor, in order to enable the surgeon OP to operate the medical observation apparatus 100 appropriately as intended, the medical observation apparatus 100 operates only when the surgeon OP appropriately grips the barrel member 122, the medical operating device, or the like. According to the present embodiment, even in a state in which the medical observation apparatus 100 is covered with the medical sterilization cover 300 or even in a situation in which the surgeon OP looks at the medical captured image, the surgeon OP can operate the medical observation apparatus 100 appropriately as intended. The embodiment of the present disclosure created by the present inventor will be described in detail below in detail.

### 4. EMBODIMENT

### 4.1. Detailed Configuration of Medical Observation Apparatus 100

A detailed configuration of the medical observation apparatus 100 according to an embodiment of the present disclosure will be described below with reference to FIGS. 3 to 5. Further, in the following description, description of the points common to the description of the schematic configuration of the medical observation system 1000 and the schematic configuration of the apparatuses of the medical observation system described above will be omitted, and only different points will be described. In detail, in the medical observation apparatus 100 according to the present embodiment, the grip detecting section 140 and the light emitting section 150 are installed on the outer circumference of the barrel member 122 of the imaging device 106. Details of the grip detecting section 140 and the light emitting section 150 will be sequentially described below.

### (4.1.1. Grip Detecting Section 140)

The grip detecting section 140 is installed on the outer circumference of the imaging device 106 and detects gripping of the imaging device 106 by the surgeon OP. Specifically, as illustrated in FIGS. 3 to 5, one or more grip detecting sections 140 are installed on the outer circumference of the barrel member 122 of the imaging device 106. Further, the grip detecting section 140 can transmit a result (grip sensing data) of detecting the gripping of the imaging device 106 by the surgeon OP to the control section 170 to be described later.

It is sufficient if the grip detecting section 140 is a sensor capable of detecting direct gripping of the imaging device 106 by the surgeon OP and gripping via the medical sterilization cover 300 and may be, for example, a capacitive type sensor that detects capacitance of a contact surface by a contact of a finger of the surgeon OP. Further, the grip detecting section 140 is not limited to a capacitive type sensor but may be, for example, a resistive film type sensor or a pressure sensitive type sensor. Further, since it can be installed without changing the structure of the imaging device 106 greatly, in order to suppress an increase in a manufacturing cost or the like of the medical observation apparatus 100 or time and efforts to change the medical observation apparatus 100, it is desirable to select the capacitive type sensor as the grip detecting section 140. Further, a grip detection level of the grip detecting section 140 can be adjusted appropriately, but it is desirable that it be adjusted so that the grip is detected when the hand of the surgeon OP grips the imaging device 106 firmly.

Further, in addition to the detection of the grip of the surgeon OP, the grip detecting section 140 can also detect a gripping position of the surgeon OP, that is, the positions of the fingers of the surgeon OP by associating the position at which the grip detecting section 140 is installed with a detection result in the grip detecting section 140. Further, the grip detecting section 140 can detect a grip strength (strong and weak) of the surgeon OP.

More specifically, the grip detecting section 140 includes a grip detecting section 140a installed on the outer circumference on the front side and the lower end side of the barrel member 122 and a grip detecting section 140b installed on the outer circumference on the back side and lower end side of the barrel member 122. For example, the grip detecting section 140a is installed at a position at which a part of the palm of the surgeon OP comes into contact with it in a state in which the surgeon OP pushes or is able to push the operating mode change switch 128 to set the free mode as illustrated in FIG. 3. Further, for example, the grip detecting section 140b is installed at a position at which the inside of the finger or a part of the palm of the surgeon OP comes into contact with it in a state in which the surgeon OP pushes or is able to push the operating mode change switch 128 to set the free mode as illustrated in FIG. 3.

In other words, the grip detecting section 140 is installed at a position at which the hand (the finger or the palm) of the surgeon OP comes into contact with (touches) it in a state where the surgeon OP pushes or is able to push the operating mode change switch 128. In other words, the position at which the grip detecting section 140 is installed or the size of the grip detecting section 140 (the size of the detection surface) is selected so that the hand of the surgeon OP can comes into contact the grip detecting section 140 when the surgeon OP grips the medical operating device or the barrel member 122 appropriately. Further, it is desirable that a plurality of grip detecting sections 140 are installed on the outer circumference of the barrel member 122 of the imaging device 106, and further, it is desirable that they are installed in a wide range of the outer circumference. Accordingly, it is possible to detect the grip state of the surgeon OP with a high degree of accuracy, and it is possible to operate the medical observation apparatus 100 more appropriately as intended.

Further, in the present embodiment, the position and the form of the grip detecting section 140 are not limited to the example illustrated in FIGS. 3 to 5, and for example, only one of the grip detecting section 140a and the grip detecting section 140b may be installed.

### (4.1.2. Light Emitting Section 150)

The light emitting section 150 is installed to overlap the outer circumference or the upper surface of the barrel member 122 of the imaging device 106 or the medical operating device and indicates the position of the medical operating device or the direction (attitude) of the imaging device 106 by turning on a light on the basis of the detection result (grip sensing data) of the grip detecting section 140. Specifically, the light emitting section 150 includes a lighting component such as a light emitting diode (LED), and is controlled such that it turns on a light in a case in which the grip of the surgeon OP is detected by the grip detecting section 140. Accordingly, even in a state in which the medical observation apparatus 100 is covered with the medical sterilization cover 300, the surgeon OP can easily recognize the position of the medical operating device and the direction (attitude) of the imaging device 106. Further, in a case in which the grip of the surgeon OP is not detected by the grip detecting section 140, the light emitting section 150 is controlled such that it turns off a light. Accordingly, it is possible to prevent the observation of the operating site from being hindered by the light of the light emitting section 150 when the surgeon OP desires to observe the operating site.

More specifically, the light emitting section 150 includes a light emitting section 150a installed overlapped with the function switch 123, the zoom switch 124, and the focus switch 126 installed on the outer circumference on the front side of the barrel member 122 of the imaging device 106 as illustrated in FIG. 4. Further, as illustrated in FIG. 5, the light emitting section 150 includes a light emitting section 150a installed overlapped with the operating mode change switch 128 installed on the outer circumference on the back side of the barrel member 122. Further, the light emitting section 150 includes a light emitting section 150c installed on the back side of the upper surface of the barrel member 122 of the imaging device 106 as illustrated in FIG. 3. The light emitting section 150a can indicate the positions of the function switch 123, the zoom switch 124, and the focus switch 126 by turning on a light. Similarly, the light emitting section 150b can indicate the position of the operating mode change switch 128 by turning on a light. Further, the light emitting section 150c can indicate the upward and downward directions of the imaging device 106 or the position of the back surface of the imaging device 106, that is, the direction (attitude) of the imaging device 106 by turning on a light.

Further, in the present embodiment, the position and the form of the light emitting section 150 are not limited to the example illustrated in FIGS. 3 to 5, but one or two of the light emitting section 150a, 150b, and 150c may be installed.

### (4.1.3. Control Section 170)

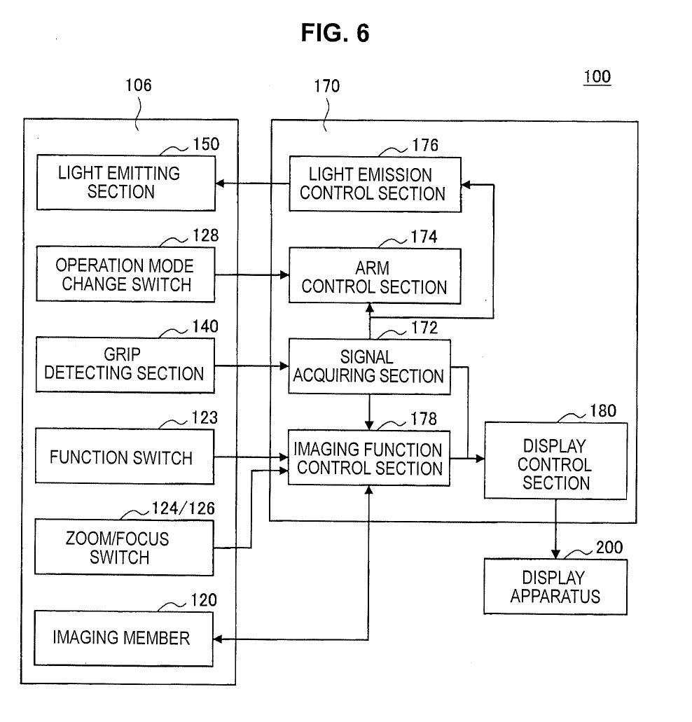

Further, in the present embodiment, the medical observation apparatus 100 includes the control section 170 which acquires sensing data or the like from the grip detecting section 140 and controls the medical observation apparatus 100. The control section 170 according to the present embodiment will be described below with reference to FIG. 6. FIG. 6 is a functional block diagram illustrating an example of a configuration of the medical observation apparatus 100 according to the present embodiment. Specifically, the main functional blocks of the imaging device 106 according to the present embodiment described above (the imaging member 120, the function switch 123, the zoom switch 124, the focus switch 126, the operating mode change switch 128, the grip detecting section 140, and the light emitting section 150) are illustrated on the left side of FIG. 6. Further, the control section 170 to be described below is illustrated on the right side of FIG. 6.

The control section 170 is constituted by, for example, the above-described processor (not illustrated) and has a function of controlling the whole medical observation apparatus 100. Further, the process related to the control method in the control section 170 may be performed by a plurality of a process circuits (for example, a plurality of processors) in a distributed manner.

In detail, the control section 170 can detect that the surgeon OP appropriately grips the imaging device 106 (specifically, the barrel member 122) on the basis of the detection result (grip sensing data) of the grip detecting section 140. Further, the control section 170 can perform control for the medical observation apparatus 100 (for example, control for the imaging device 106, the arm 104, or the like) on the basis of the detection result of the grip detecting section 140 or a detection result of an operation on the medical operating device (for example, the function switch 123, the zoom switch 124, the focus switch 126, or the operating mode change switch 128) in addition to the detection result of the grip detecting section 140. Further, the control section 170 can transfer information related to the grip state of the surgeon OP or the operating state for the medical operating device to the display apparatus 200 in real time.

More specifically, as illustrated in FIG. 6, the control section 170 includes a signal acquiring section (sensing information acquiring section) 172, an arm control section 174, a light emission control section 176, an imaging function control section 178, and a display control section 180. The respective functional blocks of the control section 170 according to the present embodiment will be sequentially described below in detail. Further, in the present embodiment, the configurations of the function blocks of the control section 170 are not limited to the example illustrated in FIG. 6, and for example, other function blocks or the like may be included.

### (Signal Acquiring Section 172)

The signal acquiring section 172 acquires the detection result (grip sensing data) of the grip detecting section 140. The signal acquiring section 172 determines whether or not the grip of the surgeon OP is an appropriate grip (the grip state) on the basis of the detection result, and outputs a determination result to the arm control section 174, the light emission control section 176, or the like. For example, in a case in which a plurality of grip detecting sections 140 are installed in the imaging device 106, the signal acquiring section 172 determines that the grip of the surgeon OP is an appropriate grip when the number of grip detecting sections 140 in which the grip is detected is a preset threshold value or more.

Further, the signal acquiring section 172 can also detect the grip strength (strong and weak) of the surgeon OP on the basis of the detection result of the grip detecting section 140, and the signal acquiring section 172 can further detect the grip position of the surgeon OP, that is, the position of the hand of the surgeon OP by associating the position at which the grip detecting section 140 is installed with the detection result in the grip detecting section 140. In this case, the signal acquiring section 172 can determine that the grip of the surgeon OP is an appropriate grip when the detected grip position corresponds to a preset position, and the grip strength is a preset threshold value or more.

### (Arm Control Section 174)

The arm control section 174 controls driving of the arm 104. As an example of the control of driving the arm 104, for example, a control signal for controlling driving can be applied to a brake (not illustrated) corresponding to each of the joint sections 110. In detail, the arm control section 174 controls the driving of the arm 104 (for example, releases the brake) on the basis of the determination result (the grip state determination result) of the signal acquiring section 172 based on the detection result of the grip detecting section 140 or the detection result of the operation input to the operating mode change switch 128 in addition to the determination result of the signal acquiring section 172. In the latter case, the arm control section 174 can determine whether the operation input of the surgeon OP is valid or invalid by combining the determination result of the signal acquiring section 172 and the detection result of the operation input to the operating mode change switch 128. More specifically, the arm control section 174 invalidates the operation input to the operating mode change switch 128 in a case in which it is determined that the grip of the surgeon OP is not appropriate on the basis of the detection result of the grip detecting section 140. More specifically, the arm control section 174 can perform control such that the brake is released in a case in which the grip detecting section 140 detects an appropriate grip of the surgeon OP. With this control, in the present embodiment, it is possible to prevent an operation of the arm 104 caused by an unintended operation input. Further, in the following description, it is assumed that the arm control section 174 controls the driving of the arm 104 on the basis of the detection result of the operation input to the operating mode change switch 128 in addition to the determination result of the signal acquiring section 172.

### (Light Emission Control Section 176)

The light emission control section 176 controls driving of the light emitting section 150 on the basis of the determination result of the signal acquiring section 172 for the detection result of the grip detecting section 140 (the grip state determination result). As an example of the control of the driving of the light emitting section 150, for example, a control signal for controlling the driving can be applied to the light emitting section 150 so that the light emitting section 150 is turned on or off. More specifically, the light emission control section 176 controls the light emitting section 150 such that it turns on a light in a case in which the gripping of the imaging device 106 by the surgeon OP is detected.

### (Imaging Function Control Section 178)

The imaging function control section 178 controls imaging conditions or the like of the imaging device 106. Examples of the control of the imaging conditions of the imaging device 106 include control of an AF function including, for example, the zoom function (the optical zoom function and the electronic zoom function), an autofocus operation, and a function of changing a frequency of illumination light irradiated from a light source. In detail, the imaging function control section 178 may control the imaging conditions or the like of the imaging device 106 on the basis of the determination result (the grip state determination result) of the signal acquiring section 172 based on the detection result of the grip detecting section 140 or the detection result of the operation input to the function switch 123, the zoom switch 124, or the focus switch 126 in addition to the determination result of the signal acquiring section 172. In the latter case, the imaging function control section 178 can determine whether the operation input of the surgeon OP is valid or invalid by combining the determination result of the signal acquiring section 172 with the detection result of the operation input to the zoom switch 124 or the like. For example, the imaging function control section 178 invalidates the operation input to the zoom switch 124, or the like in a case in which the grip of the surgeon OP is determined to be inappropriate on the basis of the detection result of the grip detecting section 140. More specifically, the imaging function control section 178 can accept the operation input to the zoom switch 124 or the like, in a case in which the grip detecting section 140 detects an appropriate grip of the surgeon OP. With this control, in the present embodiment, it is possible to prevent the imaging conditions or the like of the imaging device 106 from being controlled by an unintended operation input.

### (Display Control Section 180)

The display control section 180 controls display in the display apparatus 200 by transferring, for example, a display control signal and an image signal to a communication device (not illustrated) and causing the display control signal and the image signal to be transmitted to the display apparatus 200. Further, communication control in the communication device may be performed by a communication control section (not illustrated) included in the control section 170. Further, the display control section 180 can also control the display in the display apparatus 200 on the basis of the determination result (the grip state determination result) of the signal acquiring section 172 based on the detection result of the grip detecting section 140 or the detection result of the operation input to the function switch 123, the zoom switch 124, or the focus switch 126 and the operating mode change switch 128 in addition to the determination result of the signal acquiring section 172. Further, an example of the display image of the display apparatus 200 in the present embodiment will be described later.

Further, the functional configuration for realizing the process related to the control method to be described later in the medical observation apparatus 100 according to the present embodiment is not limited to the configuration illustrated in FIG. 6, and for example, the medical observation apparatus 100 according to the present embodiment can have a functional configuration corresponding to a method of dividing the process related to the control method. For example, the medical observation apparatus 100 according to the present embodiment may include some or all of the signal acquiring section 172, the arm control section 174, the light emission control section 176, the imaging function control section 178, and the display control section 180 separately from the control section 170 (for example, can realize them through separate processing circuits).

Further, in a case in which the medical observation system 1000 according to the present embodiment has a configuration including a medical control apparatus (not illustrated), and the medical observation apparatus 100 according to the present embodiment is controlled by the medical control apparatus, the medical observation apparatus 100 need not necessarily include the control section 170. Here, for example, the medical control apparatus can perform the process related to the control method according to the present embodiment which will be described later by including a control section having a function and a configuration similar to those of the control section 170. The medical control apparatus can control the operation of the medical observation apparatus 100 according to the present embodiment by performing communication with the medical observation apparatus 100 according to the present embodiment via a communication device installed therein or an external communication device connected thereto. Further, in a case in which the medical observation system 1000 according to the present embodiment has a configuration including the medical control apparatus, and the medical observation apparatus 100 according to the present embodiment is controlled by the medical control apparatus, the medical observation apparatus 100 according to the present embodiment may have a configuration including no some functions of the control section 170.

### 4.2. Control Method According to the Present Embodiment

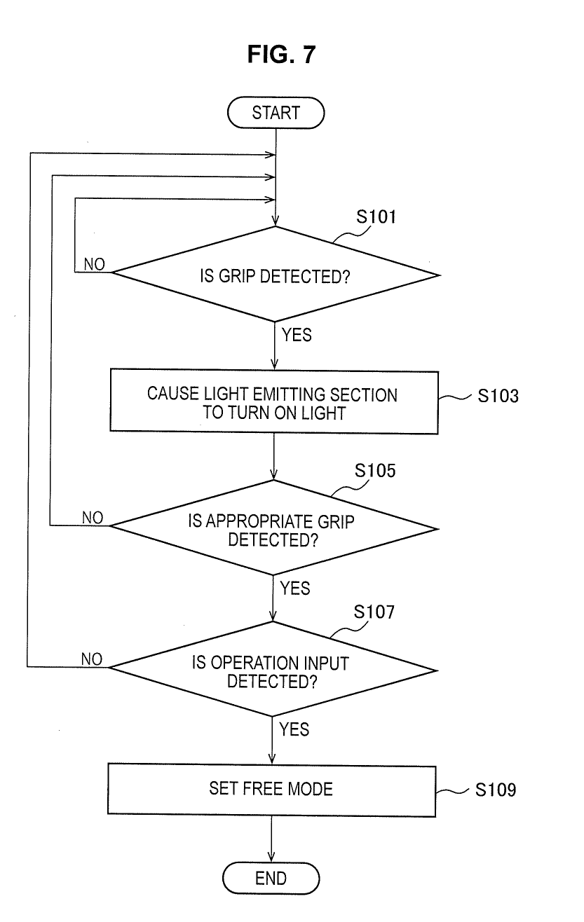

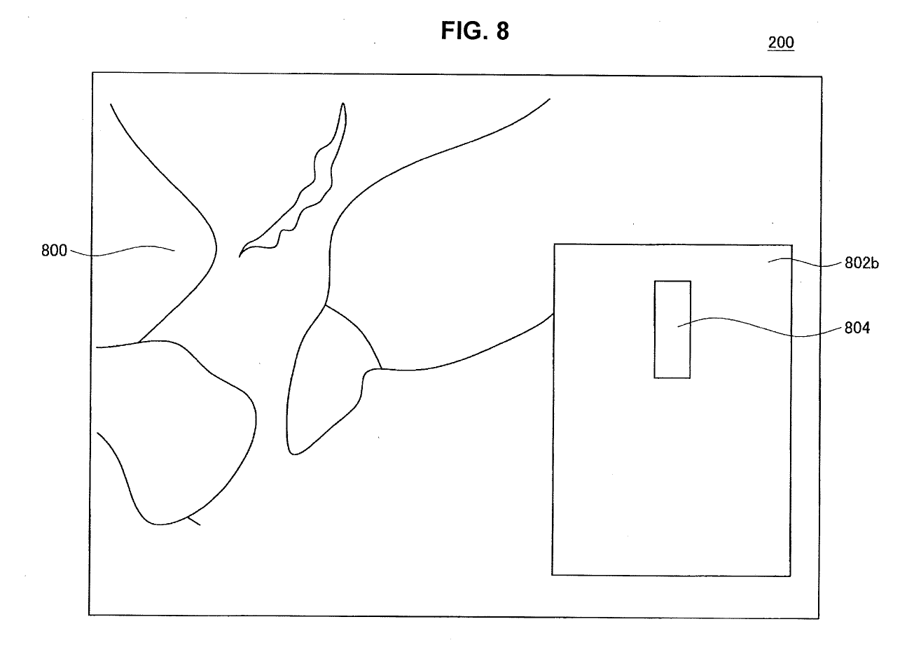

The detailed configuration of the medical observation apparatus 100 according to the present embodiment has been described above. Next, the control method according to the present embodiment will be described with reference to FIGS. 7 and 8. FIG. 7 is a flowchart illustrating an example of the process related to the control method according to the present embodiment, and FIG. 8 is an explanatory diagram illustrating an example of a display image according to the present embodiment.

As an overview of the control method according to the present embodiment, the grip of the surgeon OP is detected by the grip detecting section 140, and a detection result by the grip detecting section 140 is transferred to the control section 170. Further, the operation input to the medical operating device (for example, the operating mode change switch 128) by the surgeon OP is detected, and a detection result of the operation input to the medical operating device is also transferred to the control section 170. Then, the control section 170 determines whether the operation input to the medical operating device is valid or invalid on the basis of the combination of these detection results, and in a case in which it is determined that the operation input is valid, the control section 170 controls the medical observation apparatus 100 in accordance with the operation input. More specifically, in the present embodiment, in a case in which it is determined that the surgeon OP is not appropriately griping on the basis of the detection result by the grip detecting section 140, the operation input to the medical operating device is invalidated, and thus the malfunction of the medical observation apparatus 100 can be prevented.

In detail, the control method according to the present embodiment can include a plurality of steps from step S101 to step S109 as illustrated in the flowchart of FIG. 7. Each of steps of the control method according to the present embodiment will be described below in detail.

In the following description, it is assumed that the surgeon OP pushes the operating mode change switch 128, grips the lower side of the barrel member 122, and attempts to adjust the position and the attitude of the imaging device 106 by an operation of moving the imaging device 106 directly as illustrated by a line in FIG. 3 that alternates between long dashes and pairs of short dashes.

### (Step S101)

The medical observation apparatus 100 detects the grip of the surgeon OP by the grip detecting section 140. In a case in which the medical observation apparatus 100 determines that the surgeon OP grips it on the basis of the detection result of the grip detecting section 140, the process proceeds to step S103.

### (Step S103)

The medical observation apparatus 100 causes the light emitting section 150 to turn on a light on the basis of the grip detection result of the surgeon OP in step S101.

### (Step S105)

On the basis of the grip detection result of the surgeon OP in step S101, the medical observation apparatus 100 determines whether or not the grip is an appropriate grip, and in a case in which the grip is an appropriate grip, the process proceeds to step S107. On the other hand, in a case in which the grip is not an appropriate grip, the medical observation apparatus 100 returns to step S101, and the process is repeated from the grip detection. For example, in a case in which the grip is detected by the grip detecting section 140, the medical observation apparatus 100 can determine that the grip of the surgeon OP is an appropriate grip of gripping an appropriate position. Further, in a case in which a plurality of grip detecting sections 140 are installed, the medical observation apparatus 100 determines that the grip of the surgeon OP is an appropriate grip in a case in which the number of grip detecting section 140 which have detected the grip is a preset threshold value or more. Alternatively, the medical observation apparatus 100 may determine that the grip of the surgeon OP is an appropriate grip in a case in which the strength of the grip detected by the grip detecting section 140 is a preset threshold value or more. Further, the medical observation apparatus 100 may determine that the grip of the surgeon OP is an appropriate grip on the basis of the above combination (the position, the number, and the strength). Further, in a case in which the grip strength of the surgeon OP is a threshold value or more, and thus it is determined that the grip of the surgeon OP is an appropriate grip, it can be regarded that the grip of the surgeon OP is a stable grip, and thus the observation range of the imaging device 106 is more stably moved or fixed.

### (Step S107)

The medical observation apparatus 100 detects the operation input to the operating mode change switch 128 by the surgeon OP. In a case in which the medical observation apparatus 100 detects the operation input to the operating mode change switch 128, the process proceeds to step S109 On the other hand, in a case in which the medical observation apparatus 100 has not detected the operation input to the operating mode change switch 128, the process returns to step S101.

### (Step S109)

The medical observation apparatus 100 releases the brake (not illustrated) corresponding to each joint section 110 of the arm 104 and sets the arm 104 to the free mode. Accordingly, it is possible to adjust the position and the attitude of the imaging device 106 by the direct operation by the surgeon OP.

At this time, information of the detected operation input to the operating mode change switch 128 by the surgeon OP is displayed on the display apparatus 200 together with the medical captured image. For example, as illustrated in FIG. 8, the display apparatus 200 displays an icon 802b (grip state display image) indicating the state of the operating mode change switch 128 together with a medical captured image 800 of the operating site. In particular, the icon 802b schematically indicates the operating mode change switch 128 installed on the outer circumference on the back side of the barrel member 122 of the imaging device 106 and a surrounding form thereof. Further, in a case in which there is an operation input to the operating mode change switch 128 by the surgeon OP, a brightness, a color, or the like of a rectangular mark 804 imitating the operating mode change switch 128 which is positioned in the icon 802b changes. As described above, as the brightness, the color, or the like of the rectangular mark 804 changes, it is possible to indicate the state of the operation input by the surgeon OP, that is, the state of the operating mode change switch 128.

In the present embodiment, as the icon 802 is displayed, the surgeon OP can recognize the state of the operating mode change switch 128 while keeps his/her eyes on the display apparatus 200 displaying the medical captured image 800 of the operating site, and thus the progress of the surgery of the surgeon OP can become smoother.

Further, it is desirable that the icon 802b be displayed not to overlap the medical captured image 800 of the operating site so that the progress of the surgery by the surgeon OP is not hindered. Further, in the present embodiment, the icon 802b is not limited to the form illustrated in FIG. 8, and other forms may be used.

As described above, in the present embodiment, the medical observation apparatus 100 determines whether the operation input of the surgeon OP is valid or invalid on the basis of the combination of the determination result (the grip state determination result) of the signal acquiring section 172 based on the detection result of the grip detecting section 140 and the detection result of the operation input on the operating mode change switch 128. Then, in a case in which it is determined that the grip of the surgeon OP is appropriate on the basis of the detection result of the grip detecting section 140, the operation input is validated, and thus movement of the arm 104 by an unintended operation input can be prevented.

Until now, in the state in which the medical observation apparatus 100 is covered with the medical sterilization cover 300, the visibility of the operating mode change switch 128 of the medical observation apparatus 100 is lowered, and a part of the body of the surgeon OP may collide with the operating mode change switch 128 or the like accidentally, and the observation range of the imaging device 106 may be moved contrary to the intention of the surgeon OP. On the other hand, in the present embodiment, for example, the operation input to the operating mode change switch 128 is validated by detecting that the surgeon OP intentionally grips the barrel member 122 of the imaging device 106, that is, by detecting that an appropriate grip is performed. As a result, according to the present embodiment, it is possible to prevent the observation range of the imaging device 106 from being moved contrary to the intention of the surgeon OP. In other words, in the present embodiment, when the operation input to the operating mode change switch 128 is performed in a state in which an appropriate grip is not performed, the operation input to the operating mode change switch 128 is invalidated. As a result, according to the present embodiment, it is possible to prevent the observation range of the imaging device 106 from being moved contrary to the intention of the surgeon OP. In other words, according to the present embodiment, the medical observation apparatus 100 can be operated appropriately as intended.

Further, in the above description, the example in which the surgeon OP pushes the operating mode change switch 128 and sets it to the free mode has been described. However, the present embodiment is not limited to the application in such a case. For example, it can be applied even in a case in which the surgeon OP pushes the zoom switch 124 or the focus switch 126 and desires to adjust the imaging conditions in the imaging device 106. Further, in the present embodiment, for example, the control section such as the arm control section 174 may control the driving of the arm 104 or the like on the basis of only the determination result (grip state determination result) of the signal acquiring section 172 using the detection result of the grip detecting section 140. Alternatively, in the present embodiment, the functions of various types of medical operating devices may be switched on the basis of the grip detection result.

### 5. MODIFIED EXAMPLES

The embodiment of the present disclosure described above can be modified in various ways. In this regard, various types of modified examples of the present embodiment will be described below.

### 5.1. First Modified Example

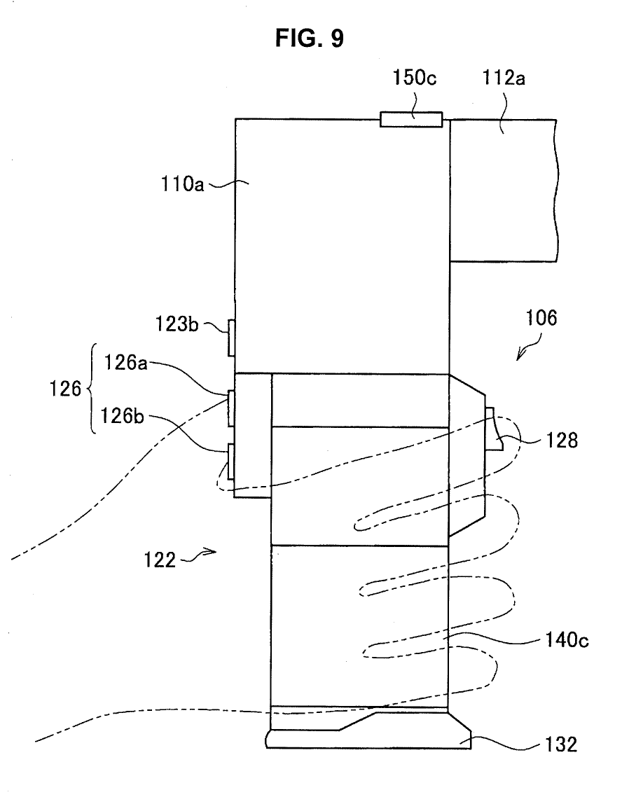

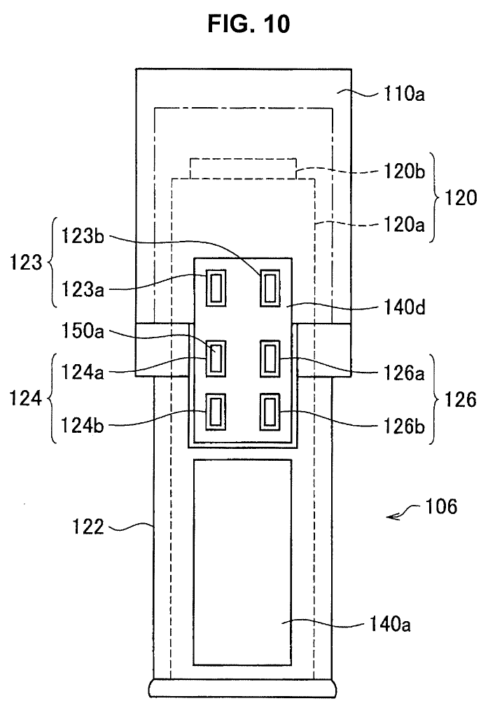

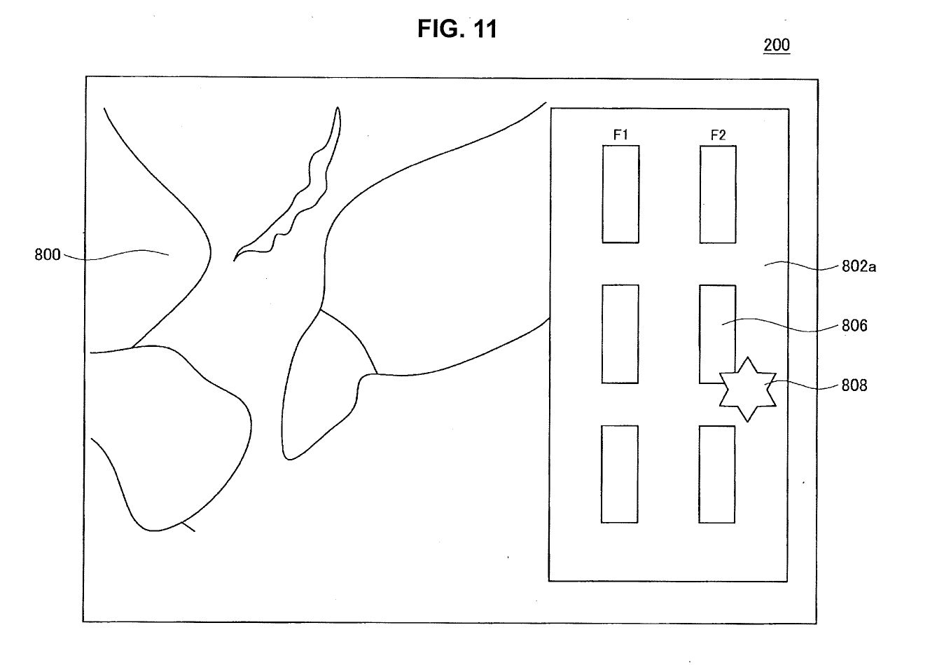

As described above, it is desirable that the grip detecting section 140 be installed on a wide range of the outer circumference of the barrel member 122 of the imaging device 106 in order to accurately detect the grip state of the surgeon OP. In this regard, a modified example of the grip detecting sections 140 which are installed on a wide range will be described with reference to FIGS. 9 to 11 as a first modified example of the embodiment of the present disclosure. FIG. 9 is an explanatory diagram for describing an example of a configuration of the imaging device 106 included in the medical observation apparatus 100 according to the present modified example, specifically, a side view of the imaging device 106 according to the present modified example. FIG. 10 is an explanatory diagram for describing another example of the configuration of the imaging device 106 included in the medical observation apparatus 100 according to the present modified example, specifically, a front view of the imaging device 106 according to the present modified example. Further, FIG. 11 is an explanatory diagram illustrating an example of a display image according to the present modified example.

Further, in the following description, description of the points common to the present embodiment described above will be omitted, and only different points will be described. In detail, the medical observation apparatus 100 according to the present modified example differs from that of the present embodiment described above in that the position and the size (the size of the detection surface) of the grip detecting section 140 are different.

As illustrated in FIG. 9, a grip detecting section 140c according to the present first modified example is installed to surround the outer circumference on the lower end side of the barrel member 122 of the imaging device 106. The grip detecting section 140c can accurately detect the position and the size (contact area) touched by the palm or the finger of the surgeon OP. Therefore, for example, the medical observation apparatus 100 can compare the strength (strong and weak) of the grip detected by the grip detecting section 140c and the size (contact area) touched by the palm and the finger of the surgeon OP with a preset threshold value and determine that the grip of the surgeon OP is an appropriate grip in a case in which the strength and the size of the grip are the threshold value or more. In other words, according to the present modified example, since the state of grip of the surgeon OP can be detected more accurately, the medical observation apparatus 100 can be operated more appropriately as intended.

Further, the grip detecting section 140 may be installed to overlap the medical operating device. In detail, as illustrated in FIG. 10, the grip detecting section 140d according to the present first modified example may be installed to overlap the function switch 123, the zoom switch 124, and the focus switch 126 of the imaging device 106. In this case, as illustrated in FIG. 10, it is desirable that the grip detecting section 140d be installed to extend to the periphery of the function switch 123, the zoom switch 124, and the focus switch 126. Accordingly, even in a case in which the hand of the surgeon OP comes into contact with a part other than the function switch 123, the zoom switch 124, and the focus switch 126, the position which the hand of the surgeon OP or the like comes into contact with can be detected. Further, in this case, the function switch 123, the zoom switch 124, and the focus switch 126 are not mechanical push buttons having a mechanically operating mechanism but may be membrane switches in which two contact sheets compete with each other with a spacer therebetween.

In other words, in a case in which the surgeon OP pushes the zoom switch 124 or the like and desires to adjust the imaging conditions in the imaging device 106, in the present modified example, it is possible to detect the contact of the hand of the surgeon OP on a part nearby the zoom switch 124 in addition to the operation input to the zoom switch 124 or the like by surgeon OP. In this regard, in the present modified example, information of the contact position of the hand of the surgeon OP on the part nearby the zoom switch 124 or the like is displayed on the display apparatus 200 together with the medical captured image 800. For example, as illustrated in FIG. 11, the display apparatus 200 displays an icon 802a (grip state display image) indicating the states of the function switch 123, the zoom switch 124, and the focus switch 126 together with the medical captured image 800 of the operating site.

In detail, the icon 802a schematically indicates the function switch 123, the zoom switch 124, and the focus switch 126 installed on the outer circumference on the front side of the barrel member 122 of the imaging device 106 and a surrounding form thereof. Further, rectangular marks 806 in the icon 802a schematically indicate the function switch 123, the zoom switch 124, and the focus switch 126. In addition, a function name F which the surgeon OP assigns the function switch 123 is displayed nearby the rectangular mark 806 schematically illustrating the function switch 123 (for example, in FIG. 11, the rectangular mark 806 positioned at the uppermost part of the icon 802a). More specifically, in the example illustrated in FIG. 11, “F1” and “F2” are displayed as the function name F assigned to the function switch 123. Then, in a case in which the hand of the surgeon OP touches a part nearby the function switch 123, the zoom switch 124, and the focus switch 126, a star-shaped mark 808 indicating the position which the hand touches is displayed on the icon 802a. Further, in a case in which the hand of the surgeon OP moves, and the contact position is moving, it is desirable that the display of the star-shaped mark 808 moves in accordance with the movement of the hand of the surgeon OP in real time. Accordingly, the surgeon OP can recognize the position of his/her hand on the function switch 123, the zoom switch 124, and the focus switch 126 even while keeping his/her eyes on the display apparatus 200 displaying the medical captured image 800 of the operating site. As a result, according to the present modified example, the surgeon OP can smoothly perform the operation input to the function switch 123, the zoom switch 124, and the focus switch 126.

Further, it is desirable that the icon 802a be displayed not to overlap the medical captured image 800 of the operating site so that the progress of the surgery by the surgeon OP is not hindered. Further, in the present modified example, the icon 802a is not limited to the form illustrated in FIG. 11, and other forms may be used.

### 5.2. Second Modified Example

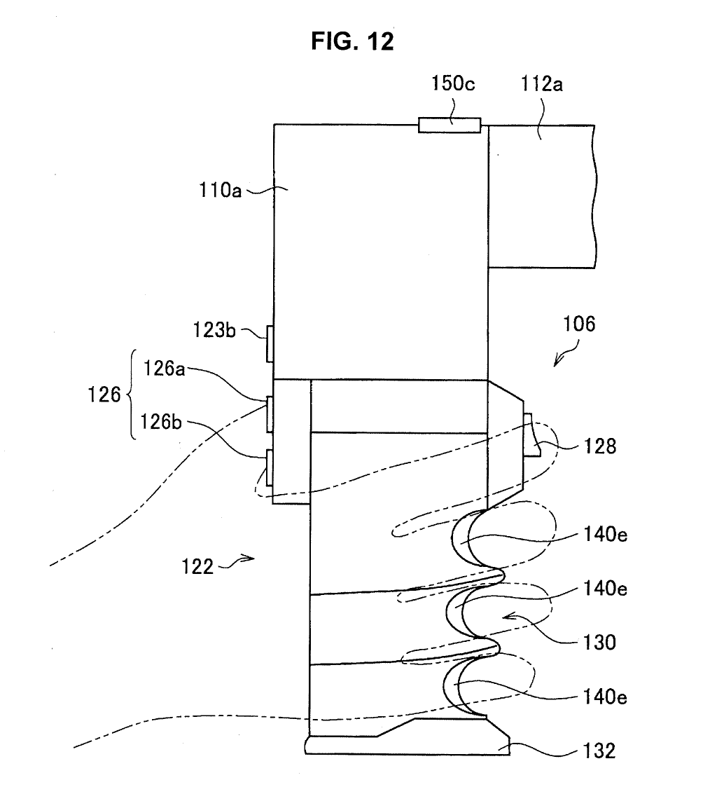

As described above, it is desirable that a plurality of grip detecting sections 140 be installed on the outer circumference of the barrel member 122 of the imaging device 106 in order to accurately detect the state of the grip of the surgeon OP. In this regard, a modified example in which a plurality of grip detecting sections 140 are installed will be described with reference to FIG. 12 as second modified example of the embodiment of the present disclosure. FIG. 12 is an explanatory diagram for describing an example of the configuration of the imaging device 106 included in the medical observation apparatus 100 according to the present modified example, specifically, a side view of the imaging device 106 according to the present modified example. Further, in the following description, descriptions of the points common to the present embodiment described above will be omitted, and only different points will be described. In detail, the medical observation apparatus 100 according to the present modified example differs from that of the present embodiment described above in that the shape of the barrel member 122 of the imaging device 106, the number of grip detecting sections 140, and the positions of the grip detecting sections 140 are different.

In detail, as illustrated in FIG. 12, on the outer circumference on the back side and the lower end side of the barrel member 122 of the imaging device 106 according to the present second modified example, a plurality of arc-like concavo-convex sections 130 are installed along the shape of the fingers at the position which the hand (the fingers and the palm) of the surgeon OP comes into contact with in the state in which the surgeon OP pushes or is able to push the operating mode change switch 128 in order to set it to the free mode. Further, a grip detecting section 140e is installed in each of a plurality of concave sections of the concavo-convex sections 130.

According to the present modified example, a plurality of arc-like concavo-convex sections 130 are installed along the shape of the fingers in the barrel member 122 of the imaging device 106, and thus it is possible to guide the surgeon OP how to grip appropriately. Further, according to the present modified example, it is possible to accurately detect contact of a plurality of fingers of the surgeon OP, that is, the grip state of the surgeon OP since the grip detecting section 140e is installed in each of the concave section.

Further, in the present modified example, the position and the form of the concavo-convex section 130 and the grip detecting section 140e are not limited to the example illustrated in FIG. 12. For example, the concavo-convex section 130 and the grip detecting section 140e may also be installed on the front side of the barrel member 122 of the imaging device 106 or may be installed to surround the outer circumference of the lower end side of the barrel member 122 of the imaging device 106.

### 5.3. Third Modified Example

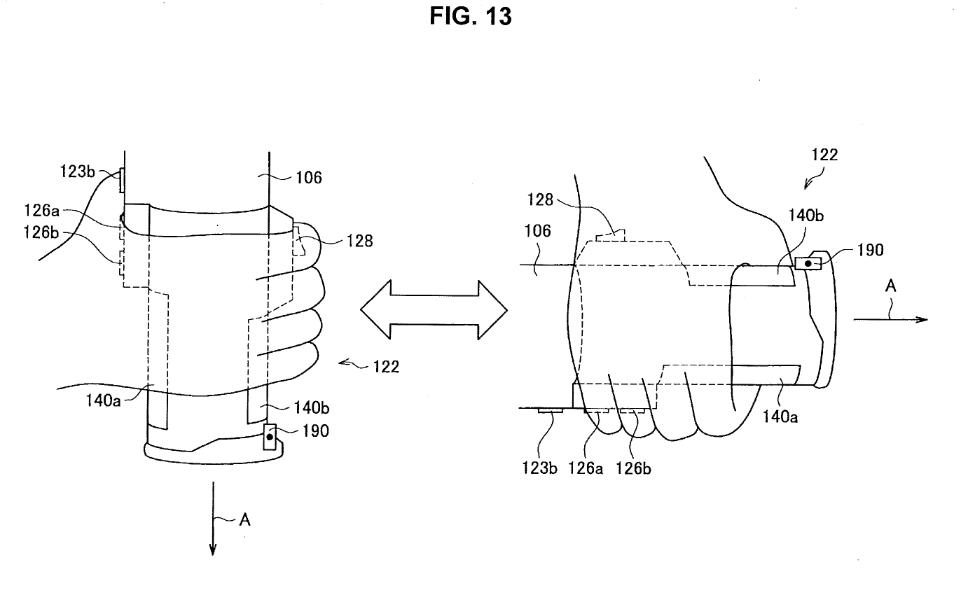

By the way, in a case in which surgeon OP desires to adjust the position and the attitude of the imaging device 106, the surgeon OP may use other grip methods instead of gripping the lower side of the barrel member 122 while pushing the operating mode change switch 128 as illustrated by a line in FIG. 3 that alternates between long dashes and pairs of short dashes to set the free mode. For example, as illustrated in FIG. 13 which is an explanatory diagram for describing a third modified example of the present embodiment and illustrates the lower end side of the barrel member 122 of the imaging device 106, a grip method, that is, an appropriate grip method may differ depending on a direction of the observation direction of the imaging device 106 (indicated by an arrow A of FIG. 13).

Specifically, for example, a case in which the lower end of the imaging device 106 faces downward along the gravitational acceleration direction as illustrated on the left side of FIG. 13, that is, a case in which the observation direction A faces downward along the gravitational acceleration direction is considered. In this case, it is desirable to grip the imaging device 106 such that the inside of the base of the thumb of the surgeon OP comes into contact with the front surface of the imaging device 106, and the inside of the base of the little finger from the index finger comes into contact with the back surface of the imaging device 106. On the other hand, for example, a case in which the lower end of the imaging device 106 faces a direction perpendicular to the gravitational acceleration direction as illustrated on the right side of FIG. 13, that is, a case in which the observation direction A is a direction perpendicular to the gravitational acceleration direction is considered. In this case, it is desirable to grip the imaging device 106 such that the inside of the base of the thumb of the surgeon OP comes into contact with the back surface of the imaging device 106, and the inside of the base of the little finger from the index finger comes into contact with the front surface of the imaging device 106. Further, the specific grip method described here is just an example of how to appropriately grip, and the present disclosure is not limited to such a grip method.

Therefore, since the appropriate grip method is different depending on the direction of the observation direction A of the imaging device 106, it is desirable for the medical observation apparatus 100 to determine whether or not the grip of the surgeon OP is an appropriate grip depending on the observation direction A of the imaging device 106. In this regard, in the present third modified example, the observation direction A of the imaging device 106 is detected, and it is determined whether or not the grip of the surgeon OP is an appropriate grip depending on the angle of the observation direction A.

First, a detailed configuration of the imaging device 106 according to the present modified example will be described with reference to FIG. 13. Further, in the following description, descriptions of the points common to the present embodiment described above will be omitted, and only different points will be described. In particular, the imaging device 106 according to the present modified example differs from that of the present embodiment described above in that an angle detecting section 190 is installed on the outer circumference of the barrel member 122 of the imaging device 106.

Specifically, as illustrated in FIG. 13, the angle detecting section 190 is installed on the outer circumference on the back side and the lower end side of the barrel member 122 of the imaging device 106, and detects the observation direction A of the imaging device 106. For example, the angle detecting section 190 can be realized by a gyro sensor, an acceleration sensor, or the like. Further, the angle detecting section 190 may be realized by a rotary encoder installed in the link 112 of the arm 104. Then, the control section 170 controls the arm 104 or the like on the basis of a detection result (angle sensing data) from the angle detecting section 190.

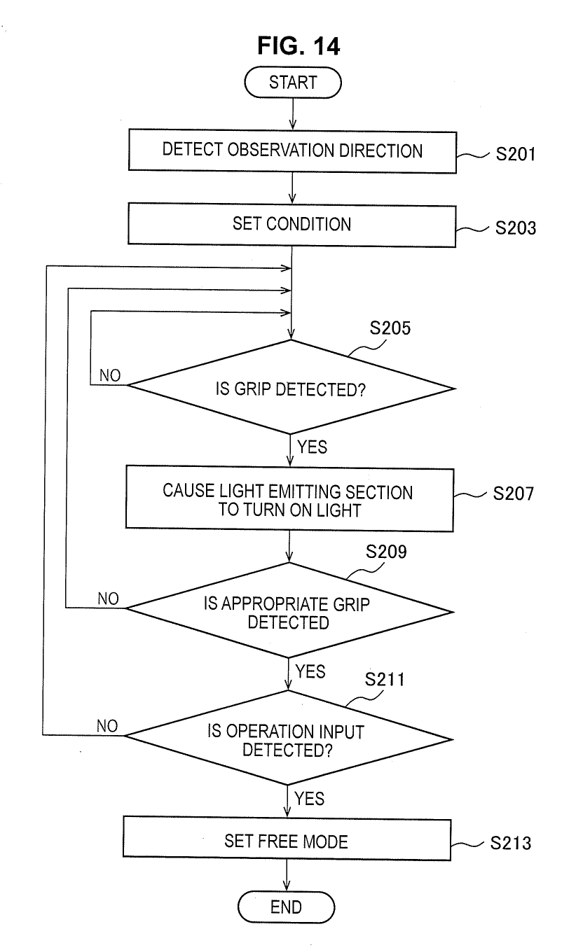

Next, a control method according to the present modified example will be described with reference to FIG. 14. FIG. 14 is a flowchart illustrating an example of a process related to the control method according to the present modified example. In detail, the control method according to the present modified example can include a plurality of steps from step S201 to step S213 as illustrated in the flowchart of FIG. 14. Each of steps of the control method according to the present modified example will be described below in detail. Further, in the following description, only points different from the present embodiment described above will be described, and description of the points common to the present embodiment will be omitted.

### (Step S201)

The medical observation apparatus 100 detects the observation direction A of the imaging device 106 by the angle detecting section 190. In detail, the medical observation apparatus 100 detects the attitude (that is, the observation direction A) of the imaging device 106 on the basis of the detection result (angle sensing data) of the angle detecting section 190 and proceeds to step S203 to be described later.

### (Step S203)

On the basis of the observation direction A of the imaging device 106 detected in step S201, the medical observation apparatus 100 sets a condition to be used when it is determined that the grip of the surgeon OP is an appropriate grip. The condition can be a threshold value which is compared with the number of grip detecting sections 140 which detect the grip of the surgeon OP, a threshold value which is compared with the grip strength of the surgeon OP, or information of a position of a grip at which the grip of the surgeon OP can be determined to be an appropriate grip. Therefore, the condition set in step S203 is used in step S209 when it is determined whether or not the grip of the surgeon OP is an appropriate grip.

### (Steps S205 to S213)

The medical observation apparatus 100 performs a process similar to steps S101 to S109 of the present embodiment illustrated in FIG. 7.

As described above, according to the present modified example, it can be determined whether or not the grip of the surgeon OP is an appropriate grip depending on the observation direction A of the imaging device 106. As a result, in the present modified example, the medical observation apparatus 100 can be operated more appropriately as intended depending on the observation direction A of the imaging device 106.

### 5.4. Fourth Modified Example

Further, a grip method, that is, an appropriate grip method may differ depending on the surgeon (operator) OP operating the medical observation apparatus 100. In this regard, in the present modified example, an appropriate grip state of each surgeon OP is registered in the medical observation apparatus 100 in advance, and the registered state is used as the condition when it is determined whether or not the grip of surgeon OP is an appropriate grip at the next time. Specifically, in the present modified example, the surgeon OP is urged to grip the imaging device 106 appropriately in advance, and information (profile information) detected by the grip detecting section 140 at that time is stored in the medical observation apparatus 100 as the above condition.

Further, in the present modified example, a detailed configuration of the medical observation apparatus 100 is common to that of the present embodiment described above, and thus description thereof is omitted here.

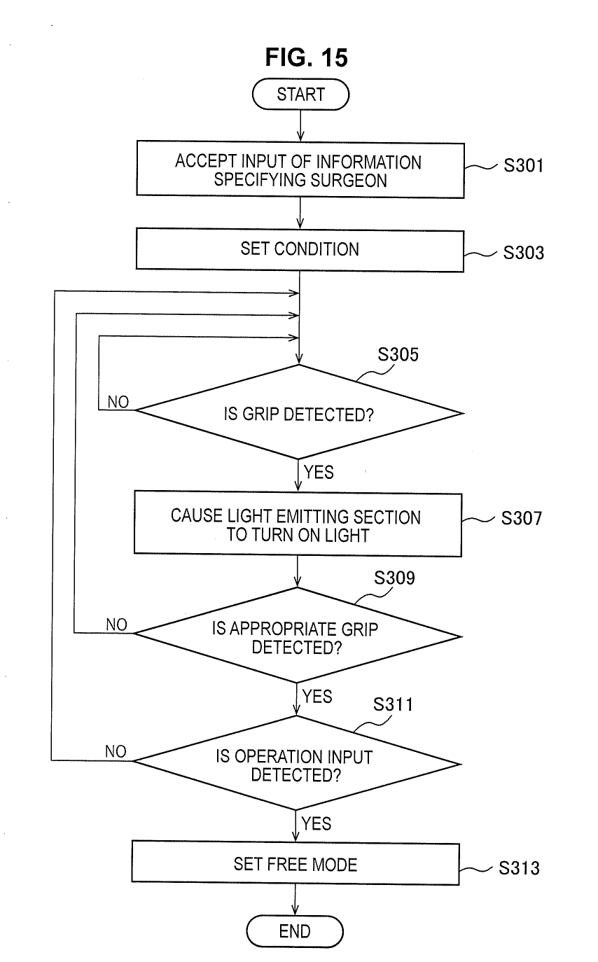

A control method according to the present modified example will be described below with reference to FIG. 15. FIG. 15 is a flowchart illustrating an example of a process related to the control method in accordance with the present fourth modified example. Specifically, the control method according to the present modified example can include a plurality of steps from step S301 to step S313 as illustrated in the flowchart of FIG. 15. Each of steps of the control method according to the present modified example will be described below in detail. Further, in the following description, only the points different from those of the present embodiment described above will be described, and description of the points common to the present embodiment will be omitted.

First, before initiating the control method according to the present modified example, as described above, the surgeon OP is urged to grip the imaging device 106 appropriately in advance, and information (profile information) such as the number of grip detecting sections 140 that detect the grip, the grip strength, and the grip position which are detected by the grip detecting section 140 at that time are registered in the medical observation apparatus 100 as the above condition.

### (Step S301)

The medical observation apparatus 100 accepts, for example, an input of information specifying the surgeon OP from the surgeon OP. For example, the information identifying the surgeon OP may be a name of the surgeon OP or may be an identification number assigned to the surgeon OP or the like.

### (Step S303)

On the basis of the information specifying the surgeon OP accepted in step S301, the medical observation apparatus 100 sets the condition used when it is determined that the grip of the surgeon OP is an appropriate grip. As described above, this condition is information (profile information) detected by the grip detecting section 140 when the surgeon OP is urged to grip the imaging device 106 appropriately in advance. Therefore, the condition set in step S303 is used in step S309 to determine whether or not the grip of surgeon OP is an appropriate grip. For example, in step S309, the medical observation apparatus 100 analyzes a correlation between the detection result of the grip detecting section 140 and the set condition, and in a case in which the detection result is a threshold value decided by the condition or more, it is determined that the grip of the surgeon OP is an appropriate grip.

### (Steps S305 to S313)

The medical observation apparatus 100 performs a process similar to steps S101 to S109 of the present embodiment illustrated in FIG. 7.

As described above, according to the present modified example, it is possible to determine whether or not the grip is an appropriate grip depending on the surgeon OP, and thus it is possible to operate the medical observation apparatus 100 more appropriately as intended.

Further, the present modified example is not limited to setting the condition used when it is determined that the grip is an appropriate grip depending on the surgeon OP, and the above condition may be set depending on, for example, a surgery technique, a position of the operating site, or a layout of the medical observation apparatus 100 or the like in the operating room.

Further, in the present modified example, the condition used when it is determined that the grip of the surgeon OP is an appropriate grip may be acquired using deep learning. For example, a plurality of detection results of the grip detecting section 140 are input to a learning device (not illustrated) when the surgeon OP feels that an appropriate and comfortable operation can be performed, and features of the detection result when the surgeon OP performs the appropriate and comfortable operation are extracted. In other words, the learning device performs supervised machine learning using a plurality of detection results of the grip detecting section 140 as input signals and using a label of an “appropriate and comfortable operation” as a teacher signal, and extracts the features of the detection results of the grip detecting section 140 in the case of the “appropriate and comfortable operation.” Further, in this case, the medical observation apparatus 100 uses the features extracted by the learning device as the condition used when it is determined that the grip of the surgeon OP is an appropriate grip. Further, the learning device may be installed in the medical observation apparatus 100 or may be installed in an apparatus outside the medical observation apparatus 100.

### 5.5. Fifth Modified Example

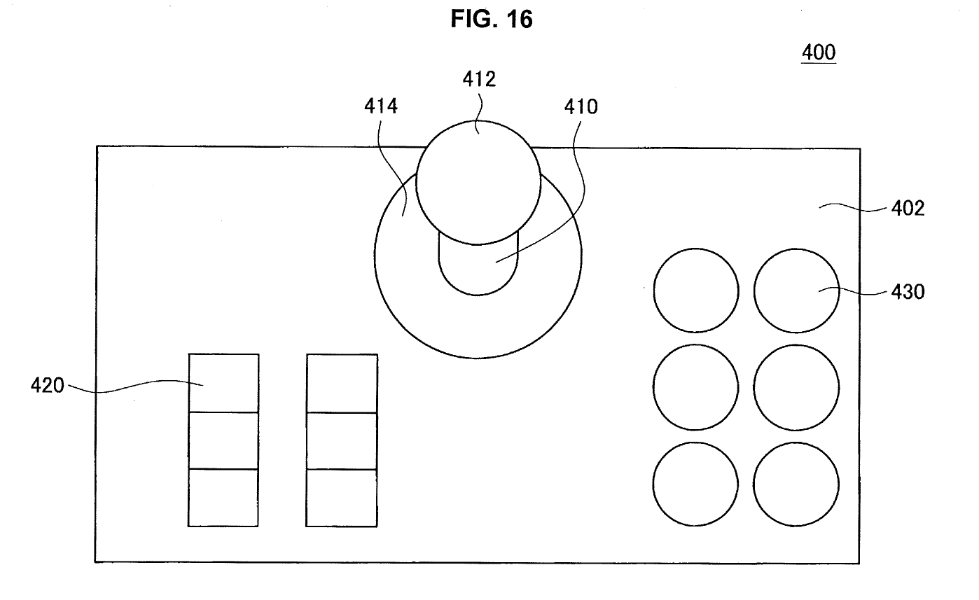

In the embodiment and the modified examples described above, the medical operating devices have been described as being the function switch 123, the zoom switch 124, the focus switch 126, and the operating mode change switch 128 installed on the outer circumference of the barrel member 122 of the imaging device 106. However, in the present embodiment and the modified examples, the medical operating device is not limited to various types of switches described above but may be, for example, a medical operating device installed separately from the medical observation apparatus 100. In this regard, an example in which the present embodiment is applied to a remote controller 400 which is a medical operating device installed separately from the medical observation apparatus 100 will be described below as a fifth modified example of the embodiment of the present disclosure. The description will proceed with reference to FIG. 16. FIG. 16 is an explanatory diagram for describing an example of a configuration of a medical operating device included in a medical observation apparatus according to the present modified example, specifically, is a schematic diagram of an example of the remote controller 400. Respective units of the hardware configuration of the remote controller 400 will be sequentially described below in detail.

As described above, the remote controller 400 according to the present modified example is an example of the medical operating device for performing the operation input to the medical observation apparatus 100 and is installed separately from the medical observation apparatus 100. For example, as illustrated in FIG. 16, the remote controller 400 includes a substantially rectangular parallelepiped housing 402, and various switches or the like for performing the operation input are installed on the upper surface of the remote controller 400. For example, a joystick (medical operating section) 410 which is a substantially rod-like member and various types of switches 420 and 430 are installed on the upper surface of the housing 402 of the remote controller 400.

In detail, as illustrated in FIG. 16, the switches 420 and 430 are switches which are installed on the upper surface of the housing 402 and have, for example, functions similar to those of the function switch 123, the zoom switch 124, the focus switch 126, and the like. Further, these zoom switches 420 and 430 may be mechanical push buttons having a mechanically operating mechanism or may be membrane switches.

For example, the joystick 410 which is a substantially rod-like member is installed to intersect perpendicularly with the upper surface of the housing 402 of the remote controller 400. The surgeon OP can drive the arm 104 or the like in accordance with the operation input, for example, by performing the operation input of inclining the joystick 410 in a desired direction. In other words, by using the joystick 410, the surgeon OP can move the observation range of the imaging device 106 by driving the arm 104 without turning his/her eyes to the joystick 410 while keeping his eyes on the display apparatus 200 displaying the medical captured image 800 of the operating site.

Further, in the present modified example, the grip detecting section 140 is installed in a head section 412 of the joystick 410, and detects gripping of the head section 412 of the joystick 410 by the surgeon OP. Alternatively, in the present modified example, the grip detecting section 140 may be installed in a peripheral section 414 on a lower side of the joystick 410 which the hand of the surgeon OP comes into contact with when the surgeon OP grips the joystick 410 and detect gripping of the joystick 410 by the surgeon OP.

Accordingly, in the present modified example, the medical observation apparatus 100 can determine whether the operation input of the surgeon OP is valid or invalid on the basis of the combination of the determination result (the grip state determination result) of the signal acquiring section 172 based on the detection result of the grip detecting section 140 and the decision result of the operation input to the joystick 410. Then, in a case in which it is determined that the grip of the surgeon OP is appropriate on the basis of the detection result of the grip detecting section 140, the operation input to the joystick 410 is validated, and thus it is possible to prevent the movement of the arm 104 caused by an unintended operation input (for example, in a case in which the joystick 410 is inclined as a part of the body of the surgeon OP touches the head section of the joystick 410 accidently).

### 6. CONCLUSION

As described above, in the embodiment and the modified examples of the present disclosure, the medical observation apparatus 100 can be operated appropriately as intended.

More specifically, according to the embodiment and the modified examples of the present disclosure, for example, the following effects can be obtained. Further, it will be appreciated that the effects obtained by the embodiment and the modified examples of the present disclosure are not limited to examples described below.

In the embodiment and the modified examples of the present disclosure, the medical observation apparatus 100 determines whether the operation input of the surgeon OP is valid or invalid on the basis of the combination of the determination result (the grip state determination result) of the signal acquiring section 172 based on the detection result of the grip detecting section 140 and the detection result of the operation input to the operating mode change switch 128. Further, according to the present embodiment and the like, in a case in which the grip of the surgeon OP is determined to be appropriate on the basis of the detection result of the grip detecting section 140, the operation input is validated, and thus it is possible to prevent the movement of the arm 104 caused by an unintended operation input.

Further, in the embodiment and the modified examples of the present disclosure, the light emitting section 150 which turns on a light in a case in which the grip intended by the surgeon OP is detected by the grip detecting section 140 is installed to overlap the medical operating device. Accordingly, according to the present embodiment and the like, even in the state in which the medical observation apparatus 100 is covered with the medical sterilization cover 300, even in a case in which the medical operating device is downsized, the surgeon OP can easily recognize the position of the medical operating device or the direction (attitude) of the imaging device 106. As a result, according to the present embodiment and the like, the malfunction of the medical observation apparatus 100 caused by an unintended operation input can be prevented, and the safety of the medical observation apparatus 100 can be enhanced.

Further, in the embodiment and the modified examples of the present disclosure, the state of the medical operating device or the contact position of the hand of the surgeon OP is displayed on the display apparatus 200 displaying the medical captured image 800 of the operating site. Accordingly, according to the present embodiment and the like, the surgeon OP can recognize the state of the medical operating device or the contact position of his/her hand even while keeping his/her eyes on the display apparatus 200, and thus the progress of the surgery by the surgeon OP can be smoother.

Further, the present embodiment and the modified examples described above are not limited to the applications to the medical observation apparatus 100 and the medical observation system 1000 but may be also applied to, for example, an endoscope, an exoscope, or a medical system including them.

Further, in the present embodiment, the surgeon OP performing the surgery has been described as operating the operating device of the medical observation apparatus 100, but the present disclosure is not limited thereto, and for example, one or more assistants supporting the surgeon OP may operate the operating device.

### 7. SUPPLEMENT

It should be understood by those skilled in the art that various modifications, combinations, sub-combinations and alterations may occur depending on design requirements and other factors insofar as they are within the scope of the appended claims or the equivalents thereof.

Further, the effects described in this specification are merely illustrative or exemplified effects, and are not limitative. That is, with or in the place of the above effects, the technology according to the present disclosure may achieve other effects that are clear to those skilled in the art from the description of this specification.

Further, respective steps in the control method of each embodiment described above may not necessarily be processed in accordance with the described order. For example, respective step may be processed in an appropriately changed order. Further, respective step may be partially processed in parallel or individually instead of being processed in a chronological order. Further, the processing method of each step need not be necessarily processed in accordance with the described method and may be processed by, for example, another method by another functional block.

Additionally, the present technology may also be configured as below.

(1) A medical observation apparatus, including:

a medical imaging section configured to image an observation target;

a support section configured to support the medical imaging section on a front end side;

at least one grip detecting section installed on an outer circumference of the medical imaging section and configured to detect a grip of a user; and

a control section configured to control the medical imaging section or the support section on the basis of a detection result of the grip detecting section.

(2) The medical observation apparatus according to (1), in which the grip detecting section detects a strength of the grip of the user or a position of a finger of the user.
(3) The medical observation apparatus according to (1) or (2), in which the control section accepts an operation input to the medical imaging section in a case in which the grip detecting section detects the grip of the user.
(4) The medical observation apparatus according to (1) or (2), in which the control section is able to release a brake of the support section in a case in which the grip detecting section detects the grip of the user.
(5) The medical observation apparatus according to (1), further including:

a sensing information acquiring section configured to acquire grip sensing data from the grip detecting section, in which the control section controls the medical imaging section or the support section on the basis of the grip sensing data.

(6) The medical observation apparatus according to (5), in which the sensing information acquiring section determines a state of the grip of the user on the basis of the grip sensing data, and

the control section controls the medical imaging section or the support section on the basis of a result of the determination.

(7) The medical observation apparatus according to (6), in which the sensing information acquiring section compares the grip sensing data with a preset predetermined threshold value, and

the control section controls the medical imaging section or the support section on the basis of a result of the comparison.

(8) The medical observation apparatus according to any one of (1) to (4), further including:

a medical operating section installed on the outer circumference of the medical imaging section,

in which the control section controls the medical imaging section or the support section on the basis of an operation input to the medical operating section.

(9) The medical observation apparatus according to (8), in which the medical operating section includes an operating button for operating the medical imaging section or an operating button for switching an operating mode of the support section.
(10) The medical observation apparatus according to (8) or (9), in which one or more grip detecting sections are installed on a part of the outer circumference which a hand of the user comes into contact with in a state in which the user is able to operate the medical operating section.
(11) The medical observation apparatus according to (10), in which an arc-like concavo-convex section is formed in at least a part of the outer circumference.
(12) The medical observation apparatus according to any one of (8) to (11), in which the grip detecting section is installed to overlap the medical operating section.
(13) The medical observation apparatus according to any one of (1) to (9), in which the grip detecting section is installed on the outer circumference to surround the medical imaging section.
(14) The medical observation apparatus according to (5), further including:

an angle detecting section configured to detect an observation direction of the medical imaging section,

in which the sensing information acquiring section determines a state of the grip of the user on the basis of the grip sensing data and angle sensing data from the angle detecting section, and

the control section controls the medical imaging section or the support section on the basis of a result of the determination.

(15) The medical observation apparatus according to (5), in which the sensing information acquiring section determines a state of the grip of the user on the basis of the grip sensing data and profile information of the user which is acquired in advance, and

the control section controls the medical imaging section or the support section on the basis of a result of the determination.

(16) The medical observation apparatus according to (8) or (9), further including:

a light emitting section configured to emit light on the basis of a detection result of the grip detecting section.

(17) The medical observation apparatus according to (16), in which the light emitting section is installed to overlap the medical operating section.
(18) The medical observation apparatus according to any one of (1) to (17), further including:

a display section configured to display a medical captured image of the observation target,

in which a grip state display image indicating a state of the grip of the user is displayed on the display section.

(19) The medical observation apparatus according to any one of (1) to (18), in which the support section includes an arm configured by connecting a plurality of links to one another via a joint section.
(20) The medical observation apparatus according to any one of (1) to (19), further including:

a medical sterilization cover configured to cover at least a part of the medical imaging section and the support section and secure a sterile state of the medical observation apparatus.

(21) A medical observation apparatus, including:

a medical imaging section configured to image an observation target;

a support section configured to support the medical imaging section on a front end side;

a substantially rod-like medical operating section configured to accept an operation input for controlling the support section;

at least one grip detecting section installed in the medical operating section and configured to detect a grip of a user; and

a control section configured to control the medical imaging section or the support section on the basis of a detection result of the grip detecting section.

What is claimed is:

1. A medical observation apparatus, comprising:
a medical imaging section configured to image an observation target;
a support section configured to support the medical imaging section on a front end side;
at least one grip detecting section installed on an outer circumference of the medical imaging section and configured to detect a grip of a user; and
a control section configured to control the medical imaging section or the support section on a basis of a detection result of the grip detecting section.

2. The medical observation apparatus according to claim 1, wherein the grip detecting section detects a strength of the grip of the user or a position of a finger of the user.

3. The medical observation apparatus according to claim 1, wherein the control section accepts an operation input to the medical imaging section in a case in which the grip detecting section detects the grip of the user.

4. The medical observation apparatus according to claim 1, wherein the control section is able to release a brake of the support section in a case in which the grip detecting section detects the grip of the user.

5. The medical observation apparatus according to claim 1, further comprising:
a sensing information acquiring section configured to acquire grip sensing data from the grip detecting section,
wherein the control section controls the medical imaging section or the support section on a basis of the grip sensing data.

6. The medical observation apparatus according to claim 5, wherein the sensing information acquiring section determines a state of the grip of the user on a basis of the grip sensing data, and
the control section controls the medical imaging section or the support section on a basis of a result of the determination.

7. The medical observation apparatus according to claim 6, wherein the sensing information acquiring section compares the grip sensing data with a preset predetermined threshold value, and
the control section controls the medical imaging section or the support section on a basis of a result of the comparison.

8. The medical observation apparatus according to claim 1, further comprising:
a medical operating section installed on the outer circumference of the medical imaging section,
wherein the control section controls the medical imaging section or the support section on a basis of an operation input to the medical operating section.

9. The medical observation apparatus according to claim 8, wherein the medical operating section includes an operating button for operating the medical imaging section or an operating button for switching an operating mode of the support section.

10. The medical observation apparatus according to claim 8, wherein one or more grip detecting sections are installed on a part of the outer circumference which a hand of the user comes into contact with in a state in which the user is able to operate the medical operating section.

11. The medical observation apparatus according to claim 10, wherein an arc-like concavo-convex section is formed in at least a part of the outer circumference.

12. The medical observation apparatus according to claim 8, wherein the grip detecting section is installed to overlap the medical operating section.

13. The medical observation apparatus according to claim 1, wherein the grip detecting section is installed on the outer circumference to surround the medical imaging section.

14. The medical observation apparatus according to claim 5, further comprising:
an angle detecting section configured to detect an observation direction of the medical imaging section,
wherein the sensing information acquiring section determines a state of the grip of the user on a basis of the grip sensing data and angle sensing data from the angle detecting section, and
the control section controls the medical imaging section or the support section on a basis of a result of the determination.

15. The medical observation apparatus according to claim 5, wherein the sensing information acquiring section determines a state of the grip of the user on a basis of the grip sensing data and profile information of the user which is acquired in advance, and
the control section controls the medical imaging section or the support section on a basis of a result of the determination.

16. The medical observation apparatus according to claim 8, further comprising:
a light emitting section configured to emit light on a basis of a detection result of the grip detecting section.

17. The medical observation apparatus according to claim 16, wherein the light emitting section is installed to overlap the medical operating section.

18. The medical observation apparatus according to claim 1, further comprising:
a display section configured to display a medical captured image of the observation target,
wherein a grip state display image indicating a state of the grip of the user is displayed on the display section.

19. The medical observation apparatus according to claim 1, wherein the support section includes an arm configured by connecting a plurality of links to one another via a joint section.

20. The medical observation apparatus according to claim 1, further comprising:
a medical sterilization cover configured to cover at least a part of the medical imaging section and the support section and secure a sterile state of the medical observation apparatus.

21. A medical observation apparatus, comprising:
a medical imaging section configured to image an observation target;
a support section configured to support the medical imaging section on a front end side;
a substantially rod-like medical operating section configured to accept an operation input for controlling the support section;
at least one grip detecting section installed in the medical operating section and configured to detect a grip of a user; and
a control section configured to control the medical imaging section or the support section on a basis of a detection result of the grip detecting section.
